(19) United States
(12) Patent Application Publication
(10) Pub. No.: US 2022/0379033 A1
(43) Pub. Date: Dec. 1, 2022

(54) RECIPROCATING MIXING AND INJECTOR SYSTEM

(71) Applicant: Windgap Medical, Inc., Watertown, MA (US)

(72) Inventors: Andrew John Ryan, Boston, MA (US); Jeffrey Thomas Chagnon, Watertown, MA (US); Phillip A. Soucy, Arlington, VA (US)

(73) Assignee: Windgap Medical, Inc., Watertown, MA (US)

(21) Appl. No.: 17/829,346

(22) Filed: May 31, 2022

Related U.S. Application Data
(60) Provisional application No. 63/194,408, filed on May 28, 2021.

(51) Int. Cl. A61M 5/20; A61J 1/20; A61M 5/315

(52) U.S. Cl. CPC A61M 5/2066; A61J 1/2089; A61J 1/2027; A61M 5/2046; A61J 1/2075; A61J 1/2037; A61M 5/31596; A61M 2005/2013; A61M 2005/206

(57) ABSTRACT

A reciprocating medicament mixing and injector system, where the energy provided to transfer medicament components back and forth between containers or cartridges can be redirected to deliver the mixed medicament components. In one embodiment the energy source is a pressurized gas chamber, in another, it is constant force spring, and in another it is a compression spring.

### CROSS-REFERENCE TO RELATED APPLICATIONS

This application claims the benefit of U.S. Provisional Patent Application No. 63/194,408 filed on May 28, 2021; which is herein incorporated by reference in entirety.

### GOVERNMENT LICENSE RIGHTS

This invention was made with government support under U01 NS112125 awarded by National Institutes of Health. The government has certain rights in the invention.

### FIELD OF THE INVENTION

The present invention relates generally to dual container devices for reconstituting or mixing medicament components.

### BACKGROUND OF THE INVENTION

Dual container/cartridge injector/autoinjectors are known for storing drug components separately until reconstitution or mixing at point of use. There are various benefits to therapeutics which may be preferred to be provided in a multi-chamber format. The drug may be more thermally stable, have a longer shelf life, or have other issues being in its aqueous form. Solubilizing drugs in liquid agents, suspending dry particles in liquids, or combining liquid-liquid solutions or suspensions thereof may be required for similar reasons.

In the field of use of multi-chambered injector/autoinjectors, there are also drug formulations where high-intensity and/or longer duration mixing is needed after recombination of the drug constituents prior to delivery of difficult to mix drug products. This may be due to low solubility of the drug, poor surface energy or wettability of a powder or microparticle for dissolution. Other needs include making a suspension of particles homogeneously dispersed within a solvent, solving problems with caking of a dry phase requiring initial energy for dispersion, or poor miscibility making emulsification difficult. In some cases, speed and ease-of-use may be critical for rescue applications where an emergency treatment needs to be delivered very quickly and with very few steps. In this field of use, state-of-the-art devices typically rely on a user shaking the drug container to mix, dissolve, or suspend the drug. Preparation can also require multiple steps that include changing out needles, or moving drug and diluent from one container to another manually. As a result of these additional user-required step, users may experience: delays in treatment time, inadequately mixed drugs, or become generally dissatisfied with the experience of using the product. In other cases, drugs may be formulated in less ideal ways where users may be required to inject a higher dose volume, endure a less comfortable dosage form, a larger than desirable delivery needle, be exposed to additional solubilizing or stabilizing agents added to the formulation, or be required to make more frequent injections. There is significant motivation to create a device that can improve upon the mixing of drugs which are otherwise difficult to solubilize, reconstitute, or suspend by re-combination alone.

The present application seeks to solve some of these identified problems as well as other problems that will become apparent to those skilled in the art.

### SUMMARY OF THE INVENTION

Several embodiments of drug mixing and drug delivery devices are disclosed herein.

In first embodiment of a mixing and drug delivery system comprises: a housing configured to hold a first container and a second container, where in the first container contains a first medicament component and the second container contains a second medicament component; a first seal associated with the first container; a second seal associated with the second container; a mixing activation mechanism; a fluid communication assembly having a fluidic channel configured to receive a first output from the mixing activation mechanism, whereupon receiving the first output from the mixing activation mechanism causes the fluid communication assembly to open, remove or otherwise pierce the first seal and the second seal and create a fluidic pathway between the first container and the second container; a mixing system configured to alternately transfer the first and second medicaments between the first and second containers during a mixing phase; a pressurized gas chamber at least partially disposed in the housing and configured to receive a second output from the mixing activation mechanism, whereupon receiving the second output causes the pressurized gas chamber to pressurize the mixing system; a mixing trigger configured to release a portion of pressurized gas that facilitates the transfer of the first and second medicaments components between the first and second containers by the mixing system, wherein the transfer between first and second containers causes the first and second medicament components to become a mixed medicament; and a needle delivery assembly configured to be in fluid communication with the first and second containers during a delivery phase.

The mixing and drug delivery system of embodiment 1, wherein the housing is formed in a T-shape, and wherein the lower portion or shaft portion of the T-shape forms a handle.

The mixing and drug delivery system of embodiment 1, wherein the mixing activation mechanism partially encloses the pressurized gas chamber.

The mixing and drug delivery system of embodiment 1, wherein the mixing system further comprises a first gas-driven plunger associated with the first container and a second gas-driven plunger associated with the second container.

The mixing and drug delivery system of embodiment 4, wherein the mixing system further comprises a multi-directional valve configured to alternate the flow of gas directed to the first and second gas-driven plungers based on user input to the mixing trigger.

The mixing and drug delivery system of embodiment 5, whereupon receiving the second output also causes the mixing system to initially drive the first gas-driven plunger to transfer the first medicament component from the first container into the second container with the second medicament component.

The mixing and drug delivery system of embodiment 5, whereupon a user depressing the mixing trigger causes a release of a portion of gas to drive either the first or second gas-driven plunger.

The mixing and drug delivery system of embodiment 7, whereupon the user releasing the mixing trigger causes the release of a portion of gas to drive either the first or second gas-driven plunger.

The mixing and drug delivery system of embodiment 7, whereupon each subsequent depressing of the mixing trigger by the user causes the release of a portion of gas to be alternately directed to drive either the first or second gas-driven plunger.

The mixing and drug delivery system of embodiment 8, whereupon each subsequent releasing of the mixing button by the user causes the release of a portion of gas to be alternately directed to drive either the first or second gas-driven plunger.

The mixing and drug delivery system of embodiment 1, wherein the fluid communication assembly further includes a fluidic transfer channel that fluidly connects the first container and the second container upon receiving the first output to the fluid communication assembly.

The mixing and drug delivery system of embodiment 11, further including a delivery seal configured to prevent fluid communication between the fluidic transfer channel and the needle assembly during the mixing phase.

The mixing and drug delivery system of embodiment 11, wherein the fluidic transfer channel and the needle assembly are configured to be in fluid communication, and wherein the needle assembly further includes a sterility barrier covering an injection end of an injection needle of the needle assembly.

The mixing and drug delivery system of embodiment 12, wherein the needle assembly further includes a needle shield configured to be a bump trigger, and a needle shield lockout mechanism configured to maintain the needle shield in an extended state after a delivery phase.

The mixing and drug delivery system of embodiment 14, further including a delivery actuation system having at least one stored energy and configured to drive the needle of the needle assembly into a user upon being activated by the bump trigger.

The mixing and drug delivery system of embodiment 5, wherein the multi-directional valve includes a vent associated with each of the first and second gas-driven plungers and configured to release pressure from either the first or second gas-driven plunger when a new portion of gas released is directed at the alternate of the first and second gas-driven plungers.

The mixing and drug delivery system of embodiment 16, further including at least one vent obstruction component.

The mixing and drug delivery system of embodiment 17, further including a vent lockout mechanism configured to move the at least one vent obstruction component in a position to block the flow of gas from exiting one of the vents of the multi-directional valve.

The mixing and drug delivery system of embodiment 18, wherein the vent lockout mechanism includes a slide actuator having at least one ramped protrusion configured to interface with the at least one vent obstruction component.

The mixing and drug delivery system of embodiment 18, wherein the vent lockout mechanism includes a camming component configured to interface with the at least one vent obstruction component.

The mixing and drug delivery system of embodiment 19, wherein the slide actuator can be configured to be pressed, pulled or slid when the mixing trigger is depressed.

The mixing and drug delivery system of embodiment 20, wherein the camming component can be configured to be pressed, pulled or slid when the mixing trigger is depressed.

The mixing and drug delivery system of embodiment 19, wherein the slide actuator can be configured to be pressed, pulled or slid when the mixing trigger is released.

The mixing and drug delivery system of embodiment 20, wherein the camming component can be configured to be pressed, pulled or slid when the mixing trigger is released.

The mixing and drug delivery system of embodiment 17, wherein the at least one vent obstruction component is configured to block the flow of gas from exiting at least one of the vents of the multi-directional valve, which prevents the transfer of medicament components between the first and second containers.

The mixing and drug delivery system of embodiment 25, whereupon creating fluid communication between the fluid communication assembly and the delivery needle assemble redirects energy associated with the pressurized to drive the medicament components disposed in either the first or second container to exit through the fluid communication assembly and out the delivery needle assembly.

A mixing and drug delivery system embodiment 27 comprising: a housing configured to hold a first container and a second container, where in the first container contains a first medicament component and the second container contains a second medicament component; a first seal associated with the first container; a second seal associated with the second container; a first plunger associated with the first container; a second plunger associated with the second container; a mixing activation mechanism; a fluid channel having two needles configured to receive a first output from the mixing activation mechanism, whereupon receiving the first output from the mixing activation mechanism causes the fluid channel to open, remove or otherwise pierce the first seal and the second seal, and create a fluidic pathway between the first container and the second container; a pre-stored energy source at least partially disposed in the housing and configured to receive a second output from the mixing activation mechanism, whereupon receiving the second output causes the pre-stored energy source to exert a force on either the first or second plunger; a mixing system configured to release a portion of the pre-stored energy source that facilitates the transfer of the first and second medicaments components between the first and second containers, wherein the transfer between first and second containers causes the first and second medicament components to become a mixed medicament; and a needle delivery assembly configured to be in fluid communication with the first and second containers during a delivery phase.

The mixing and drug delivery system of embodiment 27, wherein the mixing activation mechanism is comprised of housing that is configured to be pulled linearly and rotated, wherein a linear pull causes the first output, and wherein a rotation input causes the second output.

The mixing and drug delivery system of embodiment 27, wherein the mixing activation mechanism is comprised of housing that is configured to be rotated, wherein a rotation causes the first output and the second output.

The mixing and drug delivery system of embodiment 27, wherein the mixing system further includes a multi-directional valve.

The mixing and drug delivery system of embodiment 27, wherein the pre-stored energy source is a pressurized gas chamber.

The mixing and drug delivery system of embodiment 31, wherein the pressurized gas chamber contains permanent gas or liquid.

The mixing and drug delivery system of embodiment 27, wherein the mixing system further includes a mixing trigger.

The mixing and drug delivery system of embodiment 27, wherein the mixing system further includes a regulator.

The mixing and drug delivery system of embodiments 30 and 33, wherein the mixing system further includes a regulator.

The mixing and drug delivery system of embodiment 35, wherein pressing and releasing the mixing trigger, causes the multi-directional valve to direct pressurized gas from the regulator through alternative paths that alternate exerting a force between the first and second plungers.

The mixing and drug delivery system of embodiment 36, wherein the exerting force on the first and second plungers cause the medicament components to transfer between the first and second containers.

The mixing and drug delivery system of embodiment 37, wherein the medicament components are transferred at least 1 time.

The mixing and drug delivery system of embodiment 37, wherein the medicament components are transferred at least 2 times.

The mixing and drug delivery system of embodiment 37, wherein the medicament components are transferred more than 2 times.

The mixing and drug delivery system of embodiment 37, wherein the medicament components are transferred at least 10 times, 20 times, 40 times, or more than 100 times.

The mixing and drug delivery system of embodiment 37, further including a vent lockout mechanism.

The mixing and drug delivery system of embodiment 27, wherein the mixing activation mechanism is comprised of a pair of compressible mixing grips.

The mixing and drug delivery system of embodiment 43, wherein a first compression of the mixing grips causes the first output.

The mixing and drug delivery system of embodiment 43, wherein a first release of the mixing grips causes the second output.

The mixing and drug delivery system of embodiment 27, wherein the mixing system further includes a release mechanism configured to release a portion of stored energy.

The mixing and drug delivery system of embodiment 46, wherein the pre-stored energy source is a compression spring or a constant force spring.

The mixing and drug delivery system of embodiment 44, wherein the first output causes a direct force on the first plunger causing the first medicament component to transfer into the second container causing the first and second medicament components to become a mixed medicament.

The mixing and drug delivery system of embodiment 48, wherein releasing the mixing grips causes a release of energy from the pre-stored energy source to exert a force on the second plunger causing the mixed medicament to transfer from the second container to the first container.

The mixing and drug delivery system of embodiment 49, wherein additional compressing and releasing of the mixing grips causes the mixed medicament to transfer between the first and second containers at least 1 time.

The mixing and drug delivery system of embodiment 49, wherein additional compressing and releasing of the mixing grips causes the mixed medicament to transfer between the first and second containers at least 2 times.

The mixing and drug delivery system of embodiment 49, wherein additional compressing and releasing of the mixing grips causes the mixed medicament to transfer between the first and second containers more than 2 times.

The mixing and drug delivery system of embodiment 49, wherein additional compressing and releasing of the mixing grips causes the mixed medicament to transfer between the first and second containers at least 10 times, 20 times, 40 times, or more than 100 times.

The mixing and drug delivery system of embodiment 27, wherein the mixing activation mechanism is comprised of a lever that is configured to be extended away from the housing, wherein an extension of the lever causes the first output.

The mixing and drug delivery system of embodiment 54, wherein a first compression of the lever causes the first medicament component in the first container to transfer to the second container causing the first and second medicament to become a mixed medicament.

The mixing and drug delivery system of embodiment 55, wherein a second extension of the lever causes the second output and a transfer of the mixed medicament from the second container to the first container.

The mixing and drug delivery system of embodiment 55, wherein the mixing system further includes a horizontal rack, pinion gear and vertical rack.

The mixing and drug delivery system of embodiment 57, further includes a rotary lock.

The mixing and drug delivery system of embodiment 55, further including a sliding lock configured to prevent an extension of the lever.

The mixing and drug delivery system of embodiment 59, wherein the sliding lock is initially coupled to a safety cap, and upon removal of the safety cap causes the sliding lock to reposition and prevent the lever from extending.

The mixing and drug delivery system of embodiment 56, wherein additional compressions and extensions of the lever cause the mixed medicament to transfer between the first and second containers at least 1 time.

The mixing and drug delivery system of embodiment 56, wherein additional compressions and extensions of the lever cause the mixed medicament to transfer between the first and second containers at least 2 times.

The mixing and drug delivery system of embodiment 56, wherein additional compressions and extensions of the lever cause the mixed medicament to transfer between the first and second containers more than 2 times.

The mixing and drug delivery system of embodiment 56, wherein additional compressions and extensions of the lever cause the mixed medicament to transfer between the first and second containers at least 10 times, 20 times, 40 times, or more than 100 times.

A mixing and drug delivery system embodiment 65 comprising: a housing configured to hold a first container and a second container, where in the first container contains a first medicament component and the second container contains a second medicament component; a first seal associated with the first container; a second seal associated with the second container; a first plunger associated with the first container; a second plunger associated with the second container; a mixing activation mechanism; a fluid channel having two needles configured to receive a first output from the mixing activation mechanism, whereupon receiving the first output from the mixing activation mechanism causes the fluid channel to open, remove or otherwise pierce the first seal and the second seal, and create a fluidic pathway between the first container and the second container; a pre-stored energy source at least partially disposed in the housing and configured to receive a second output from the mixing activation mechanism, whereupon receiving the second output causes the pre-stored energy source to exert a force on either the first or second plunger; and a mixing system configured to release a portion of the pre-stored energy source that facilitates the transfer of the first and second medicaments components between the first and second containers, wherein the transfer between first and second containers causes the first and second medicament components to become a mixed medicament.

A mixing and drug delivery system embodiment 66 comprising: a housing configured to hold a first container and a second container, where in the first container contains a first medicament component and the second container contains a second medicament component; a first seal associated with the first container; a second seal associated with the second container; a mixing activation mechanism; a mixing system having a mixing grip assembly that comprises a first grip that is stationary and extending from the housing and a second grip that is movable axially along a portion of the housing, wherein the first and second grips of the mixing grip assembly are configured to be compressed upon removing the mixing activation mechanism; a fluid communication assembly configured to receive a first output from the mixing system, whereupon receiving the first output from the mixing system causes the fluid communication assembly to open, remove or otherwise pierce the first seal and the second seal and connect a fluid pathway between the first and second containers; and a needle delivery system configured to be in fluid communication with the first and second containers during a delivery phase.

The mixing and drug delivery system comprising of embodiment 66, wherein the mixing system further includes: a first plunger associated with the first container and a second plunger associated with the second container, a first plunger rod, a second plunger rod, a mechanically regenerative energy source, and a release mechanism, and wherein the first plunger rod is in direct mechanical communication with the second grip.

The mixing and drug delivery system of embodiment 67, further including a flange associated with the second grip, which is configured to interface with and laterally translate the release mechanism.

The mixing and drug delivery system of embodiment 68, wherein the release mechanism includes a ramped portion that interfaces with the flange.

The mixing and drug delivery system of embodiment 68, wherein the release mechanism includes a ledge portion that interferingly engages with a notched portion of the second plunger rod to initially prevent the second plunger rod from moving into the second container.

The mixing and drug delivery system of embodiment 67, wherein the mechanically regenerative energy source is configured to decompress or extend the first and second mixing grips, as the mechanically regenerative energy source drives the second plunger rod into the second container, which transfers the first and second medicaments components now in a mixed medicament form into the first container, which applies a pressure on the first plunger and first plunger rod, which in turn applies a force on the second grip causing it to separate away from the first grip.

The mixing and drug delivery system of embodiment 71, wherein the first and second grips are configured to put energy back into the mechanically regenerative energy source through a user compressing the grips together once the release mechanism has been laterally translated to allow axial movement of the second plunger rod.

The mixing and drug delivery system of embodiment 72, wherein the mechanically regenerative energy source is one of a compression spring or constant force spring.

The mixing and drug delivery system of embodiment 66, wherein the mixing activation mechanism is a release pin.

The mixing and drug delivery system of embodiment 66, wherein the mixing activation mechanism is a safety release disposed between the first and second grips.

The mixing and drug delivery system of embodiment 72, wherein stored energy associated with the mechanically regenerative energy source can be redirected to force the mixed medicament from the second container through the needle delivery system, upon the needle delivery system becoming in fluid communication with the fluid communication assembly through piercing or otherwise removing a delivery septum, while maintaining the first and second grips in a compressed state.

The mixing and drug delivery system of embodiment 76, wherein the needle delivery system includes a removable needle sheath.

The mixing and drug delivery system of embodiment 76, wherein the needle delivery system can be axially translated into the fluid communication assembly.

The mixing and drug delivery system of embodiment 76, wherein the needle delivery system can further have a needle shield assembly disposed about the needle delivery system.

The mixing and drug delivery system of embodiment 67, further including an engagement flange attached to the first plunger rod, which is configured to interface with and laterally translate the release mechanism.

A drug mixing system that can be attached to an injector embodiment 81 comprising:

a housing configured to hold a first container and a second container, where in the first container contains a first medicament component and the second container contains a second medicament component; a first seal associated with the first container; a second seal associated with the second container; a mixing activation mechanism; a mixing system having a mixing grip assembly that comprises a first grip that is stationary and extending from the housing and a second grip that is movable axially along a portion of the housing, wherein the first and second grips of the mixing grip assembly are configured to be compressed upon removing the mixing activation mechanism; and a fluid communication assembly configured to receive a first output from the mixing system, whereupon receiving the first output from the mixing system causes the fluid communication assembly to open, remove or otherwise pierce the first seal and the second seal and connect a fluid pathway between the first and second containers.

A drug mixing system that can be attached to an injector embodiment 82 comprising: a housing configured to hold a first container and a second container, where in the first container contains a first medicament component and the second container contains a second medicament component; a first seal associated with the first container; a second seal associated with the second container; a mixing activation mechanism; a mixing system having a regenerative energy source and a mixing grip assembly that comprises a first grip that is stationary and extending from the housing and a second grip that is movable axially along a portion of the housing, wherein the first and second grips of the mixing grip assembly are configured to be compressed upon removing the mixing activation mechanism; and a fluid communication assembly configured to receive a first output from the mixing system, whereupon receiving the first output from the mixing system causes the fluid communication assembly to open, remove or otherwise pierce the first seal and the second seal and connect a fluid pathway between the first and second containers.

A drug mixing and injector system embodiment 83 comprising: a housing configured to hold a first container and a second container, wherein the first container contains a first medicament component and the second container contains a second medicament component; a first seal associated with the first container; a second seal associated with the second container; a first plunger rod associated with the first container that is mechanically connected to a vertical rack that is mechanically driven by a pinion gear assembly; a second plunger rod associated with the second container that is mechanically connected to a regenerative energy source; a mixing system including a lever configured to pivot about the housing; a fluid communication assembly configured to receive a first output from the mixing system, whereupon receiving the first input from the mixing system causes the fluid communication assembly to open, remove or otherwise pierce the first seal and the second seal and connect a fluid pathway between the first and second containers; and a needle delivery system configured to be in fluid communication with the first and second containers via the fluid communication assembly during a delivery phase.

The drug mixing and injector system of embodiment 83, wherein the mixing system further includes a rotatable horizontal rack coupled to the lever.

The drug mixing and injector system of embodiment 84, wherein the lever further includes a camming surface that upon pivoting the lever about the housing causes a first output where the camming surface engages the fluid communication assembly and creates fluid communication between the first and second containers.

The drug mixing and injector system of embodiment 84, further including a rotary lock in mechanical communication with the second plunger rod that prevents the second plunger rod from axially moving within the second container until rotated.

The drug mixing and injector system of embodiment 86, wherein the rotary lock includes a keyed portion that is configured to rotate off a ledge formed in the second plunger rod and into a channel formed in the second plunger rod when the horizontal rack interfaces with the camming surface and causes the rotary lock to rotate.

The drug mixing and injector system of embodiment 86, wherein the horizontal rack of the mixing system is configured to interface with a camming surface of the rotary lock that enables the horizontal rack to rotate the rotatory lock, which enables axially movement of the second plunger rod.

The drug mixing and injector system of embodiment 88, wherein the regenerative energy source is configured to release a portion of energy to drive the second plunger rod into the second container and cause a transfer of medicament components in the second container to move into the first container, whereby a force is generated on the first plunger rod, which in turn causes the vertical rack to rotate the pinion gear assembly, which in turn causes the horizontal rack to translate laterally and cause the lever to pivot about the housing.

The drug mixing and injector system of embodiment 89, wherein the regenerative energy source is configured to receive and temporarily store energy when the lever is compressed into the housing reversing the mechanical transaction occurring.

The drug mixing and injector system of embodiment 84, wherein the mixing system further includes a torsional spring coupled to the horizontal rack, and wherein the torsional spring causes the horizontal rack to rack to rotate from a vertical position when stowed to a horizontal position that engages with the pinion gear assembly when the lever is initially pivoted away from the housing.

The drug mixing and injector system of embodiment 91, whereupon the horizontal rack engaging with the pinion gear assembly enables input to the mixing lever to drive the pinion gear assembly which in turn drives the vertical rack, which drives the first plunger rod into the first container causing the first medicament component to transfer from the first container to the second container to form a mixed medicament with the second medicament component.

The drug mixing and injector system of embodiment 83, further including a sliding lock that is configured to prevent the lever from pivoting when the sliding lock is engaged.

The drug mixing and injector system of embodiment 93, further including a safety cap removably connected to the housing and configured to cover at least a portion of the delivery needle assembly, wherein the safety cap further includes an extension arm configured to engage the sliding lock and cause it to translate axially when the safety cap is removed from the housing.

A drug mixing system that can be attached to an injector embodiment 95 comprising: a housing configured to hold a first container and a second container, wherein the first container contains a first medicament component and the second container contains a second medicament component; a first seal associated with the first container; a second seal associated with the second container; a first plunger rod associated with the first container that is mechanically connected to a vertical rack that is mechanically driven by a pinion gear assembly; a second plunger rod associated with the second container that is mechanically connected to a regenerative energy source; a mixing system including a lever configured to pivot about the housing; and a fluid communication assembly configured to receive a first output from the mixing system, whereupon receiving the first input from the mixing system causes the fluid communication assembly to open, remove or otherwise pierce the first seal and the second seal and connect a fluid pathway between the first and second containers.

A drug mixing and injector system embodiment 96 comprising: a housing configured to hold a first container and a second container, wherein the first container contains a first medicament component and the second container contains a second medicament component; a first seal associated with the first container; a second seal associated with the second container; a first plunger rod associated with the first container; a second plunger rod associated with the second container that is mechanically connected to a regenerative energy source; a mixing system including a lever configured to pivot about the housing and configured to provide input energy to the regenerative energy source; a fluid communication assembly configured to receive a first output from the mixing system, whereupon receiving the first input from the mixing system causes the fluid communication assembly to open, remove or otherwise pierce the first seal and the second seal and connect a fluid pathway between the first and second containers; and a needle delivery system configured to be in fluid communication with the first and second containers via the fluid communication assembly during a delivery phase.

A drug mixing and injector system embodiment 97 comprising: a housing configured to hold a first container and a second container, wherein the first container contains a first medicament component and the second container contains a second medicament component; a first seal associated with the first container; a second seal associated with the second container; a first plunger rod associated with the first container; a second plunger rod associated with the second container; a rotary lock disposed about the second plunger rod; a mixing system including a lever configured to pivot about the housing; a fluid communication assembly configured to receive a first output from the mixing system, whereupon receiving the first input from the mixing system causes the fluid communication assembly to open, remove or otherwise pierce the first seal and the second seal and connect a fluid pathway between the first and second containers; and a needle delivery system configured to be in fluid communication with the first and second containers via the fluid communication assembly during a delivery phase.

A drug mixing and injector system embodiment 98 comprising: a housing configured to hold a first container and a second container, wherein the first container contains a first medicament component and the second container contains a second medicament component; a first seal associated with the first container; a second seal associated with the second container; a first plunger rod associated with the first container; a second plunger rod associated with the second container; a mixing system including a lever configured to pivot about the housing; a fluid communication assembly configured to receive a first output from the mixing system, whereupon receiving the first input from the mixing system causes the fluid communication assembly to open, remove or otherwise pierce the first seal and the second seal and connect a fluid pathway between the first and second containers; and a needle delivery system configured to be in fluid communication with the first and second containers via the fluid communication assembly during a delivery phase.

A drug mixing and injector system embodiment 99 comprising: a housing configured to hold a first container and a second container, wherein the first container contains a first medicament component and the second container contains a second medicament component; a first seal associated with the first container; a second seal associated with the second container; a first plunger rod associated with the first container; a second plunger rod associated with the second container that is mechanically connected to a regenerative energy source; a mixing system including a lever configured to pivot about the housing and configured to provide input energy to the regenerative energy source; a fluid communication assembly configured to receive a first output from the mixing system, whereupon receiving the first input from the mixing system causes the fluid communication assembly to open, remove or otherwise pierce the first seal and the second seal and connect a fluid pathway between the first and second containers; a sliding lock configured to prevent the lever from pivoting during a delivery phase; and a needle delivery system configured to be in fluid communication with the first and second containers via the fluid communication assembly during a delivery phase.

These embodiments and others are described in further detail below.

### BRIEF DESCRIPTION OF THE DRAWINGS

The foregoing and other objects, features, and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A-F illustrate various views of a gas-driven reciprocating mixing and injector system;

FIG. 1G illustrates a cross-sectional view of the gas-driven reciprocating mixing and injector system of FIGS. 1A-F;

FIGS. 2A.1-2B.2 illustrate various exposed views of the gas-driven reciprocating mixing and injector system demonstrating engaging a fluid communication system using a mixing activation mechanism;

FIGS. 2C.1-2D.2 illustrate various exposed views of the gas-driven reciprocating mixing and injector system demonstrating activating the gas chamber using the mixing activation mechanism;

FIGS. 3A-B illustrate an alternative gas-driven reciprocating mixing and injector system embodiment where the mixing activation mechanism provides multiple outputs using a single input;

FIGS. 4A-D illustrate various phases of a mixing trigger of a gas-driven reciprocating mixing and injector system;

FIGS. 5A-D illustrate various phases of a multi-directional valve of a gas-driven reciprocating mixing and injector system;

FIGS. 6A-E illustrate various states of a gas-driven reciprocating mixing and injector system and the transferring of the medicament components between cartridges/containers;

FIGS. 7A-D illustrate an alternative variation of the various states of a gas-driven reciprocating mixing and injector system and the transferring of the medicament components between cartridges/containers where the valve stem of the multi-directional valve has alternative starting position;

FIGS. 8A-B illustrate various views of one embodiment of a multi-directional valve venting locking mechanism for use with a gas-driven reciprocating mixing and injector system;

FIGS. 9A-B illustrate various views of an alternative embodiment of a multi-directional valve venting locking mechanism for use with a gas-driven reciprocating mixing and injector system;

FIGS. 10A-C illustrate various views of an embodiment of an inline hand compression reciprocating mixing and injector system;

FIGS. 10D-E illustrate cross-sectional views of the inline hand compression reciprocating mixing and injector system of FIGS. 10A-C;

FIG. 10F illustrates removal of a safety/activation release component of the inline hand compression reciprocating mixing and injector system of FIGS. 10A-C;

FIGS. 10G-H illustrate the phases of creating fluid communication between containers and transferring a medicament from one container to another;

FIGS. 10I-L illustrate various views of releasing the rod sliding lock from a locking position to an unlocked position;

FIGS. 10M-Q illustrate various phases of the medicament components from a ready-to-mixed phase to a delivered phase including the extension of a needle shield after the injection phase;

FIGS. 11A-C illustrate various views of an alternative embodiment of an inline hand compression reciprocating mixing and injector system;

FIG. 11D illustrates a cross-sectional perspective view of the inline hand compression reciprocating mixing and injector system of FIGS. 11A-C;

FIG. 11E illustrates removal of a safety/activation release component of the inline hand compression reciprocating mixing and injector system of FIGS. 11A-C;

FIGS. 11F-G illustrate the phases of creating fluid communication between containers;

FIGS. 11H-I illustrate various views of releasing the rod sliding lock from a locking position to an unlocked position;

FIGS. 11J-K illustrate various transfer states between containers once the spring-driven plunger rod is activated;

FIGS. 11L-N illustrate various phases of the inline hand compression reciprocating mixing and injector system of FIGS. 11A-C preparing to deliver and delivering a mixed medicament;

FIGS. 12A-B illustrate various views of an alternative embodiment of a compression lever reciprocating mixing and injector system using a rack and pinion system;

FIG. 12C illustrates a cross-sectional view of the compression lever reciprocating mixing and injector system using a rack and pinion system of FIGS. 12-B;

FIGS. 12D-E illustrate various views of the compression lever reciprocating mixing and injector system in a stowed state;

FIGS. 12F-H illustrate various views demonstrating creating fluid communication between the containers and activating the reciprocating mixing system;

FIGS. 12I-L illustrate various views of one embodiment of the horizontal rack components for use with the compression lever reciprocating mixing and injector system;

FIGS. 12M-P illustrate various views of an alternative embodiment of the horizontal rack components for use with the compression lever reciprocating mixing and injector system;

FIGS. 12Q-T illustrate various views and states of a rotary rod lock for use with a reciprocating mixing and injector system;

FIGS. 12U-W illustrate various views demonstrating rotating the rotary rod lock and releasing the plunger rod associated with the constant force spring;

FIGS. 12X-AA illustrate various views demonstrating various phases of the medicament components from a ready-to-mixed phase to a ready-to-be delivered phase;

FIGS. 12BB-GG illustrate various views demonstrating initiating the mixing lever sliding lock prior to delivering the mixed medicament components.

### DETAILED DESCRIPTION OF THE INVENTION

To provide clarity, the applicants would like to provide context around certain terms used throughout this description that is in addition to their ordinary meaning.

Distal or distal end primarily refers to the end of the mixing and injector system having the components and features to drive the plungers. In contrast, proximal or proximal end refers to the end of the device where the plungers are being driven into. For example, in all of the embodiments disclosed the delivery needle is disposed on the proximal end of the mixing and injector systems. Additionally, the distal end of the delivery needle is the end that is receiving the mixed medicament components, whereas the proximal end of the delivery needle is injecting the mixed medicament components into a recipient or otherwise releasing the mixed medicament components.

For purposes of this application the term container can include any component that is configured to hold a volume. For example, a cartridge, pre-filled syringe, a vial and so forth would be considered a container. Containers can have attachment points, removable or pierceable seals associated with them and have medicament components stored therein.

As noted, there is a need to improve upon drug mixing devices to allow for drug formulations where high-intensity and/or long duration mixing is needed after combination of the drug constituents. The inventors, who created the embodiments herein, have provided solutions to at least this noted problem as well as other problems that will become apparent upon reading this description.

In many of the embodiments provided herein there is provided a fluid communication system, that includes a pair of mixing needles, a fluidic channel and a frame. This system can be positioned in the housing in a fixed manner, where other systems engage into it, or it can movable in a distal and/or proximal manner to engage with the containers as well as needle delivery system. Greater detail and examples of this fluid communication system can be found in U.S. published application US2022/0001112 A1.

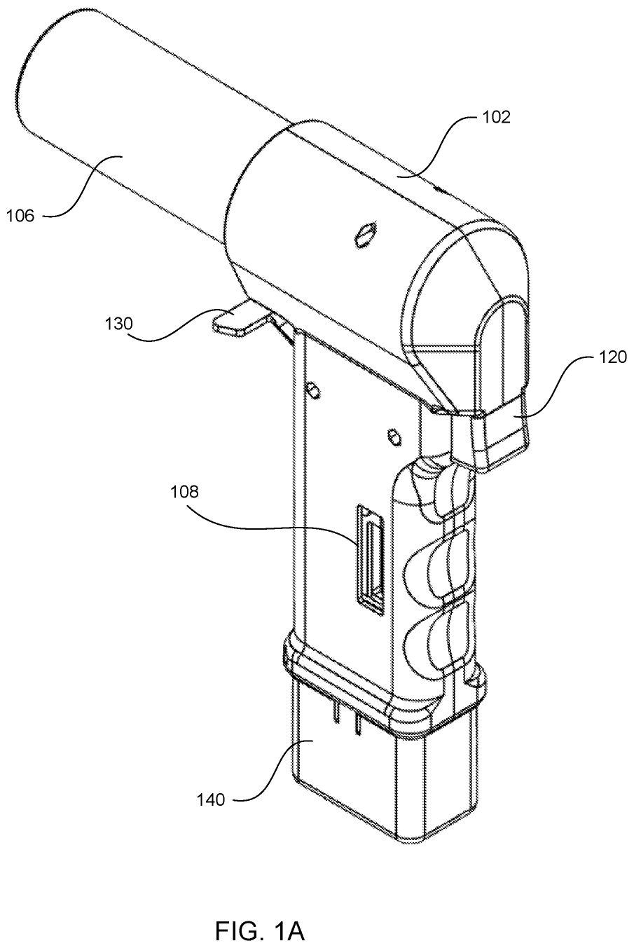

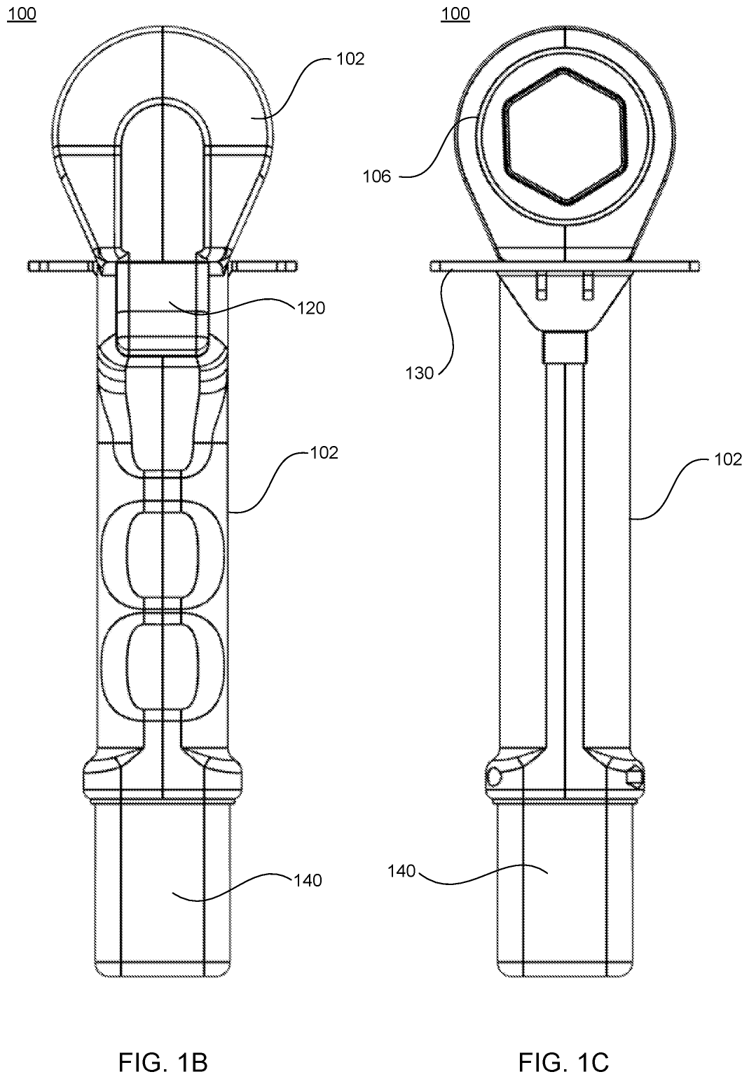

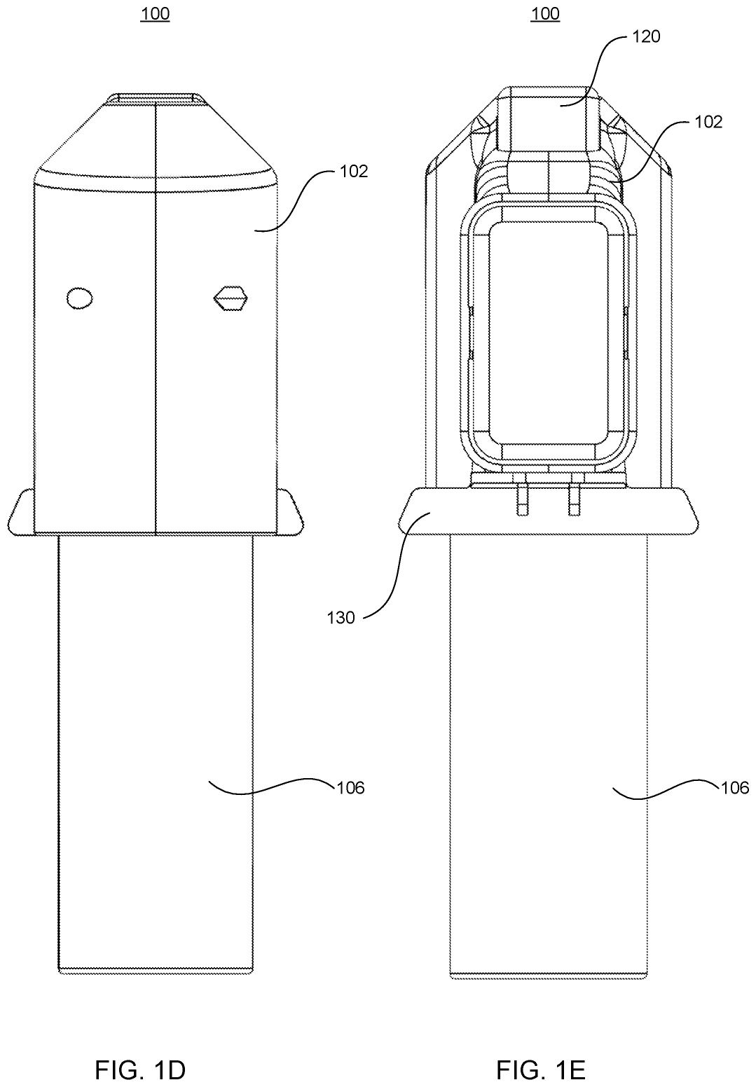

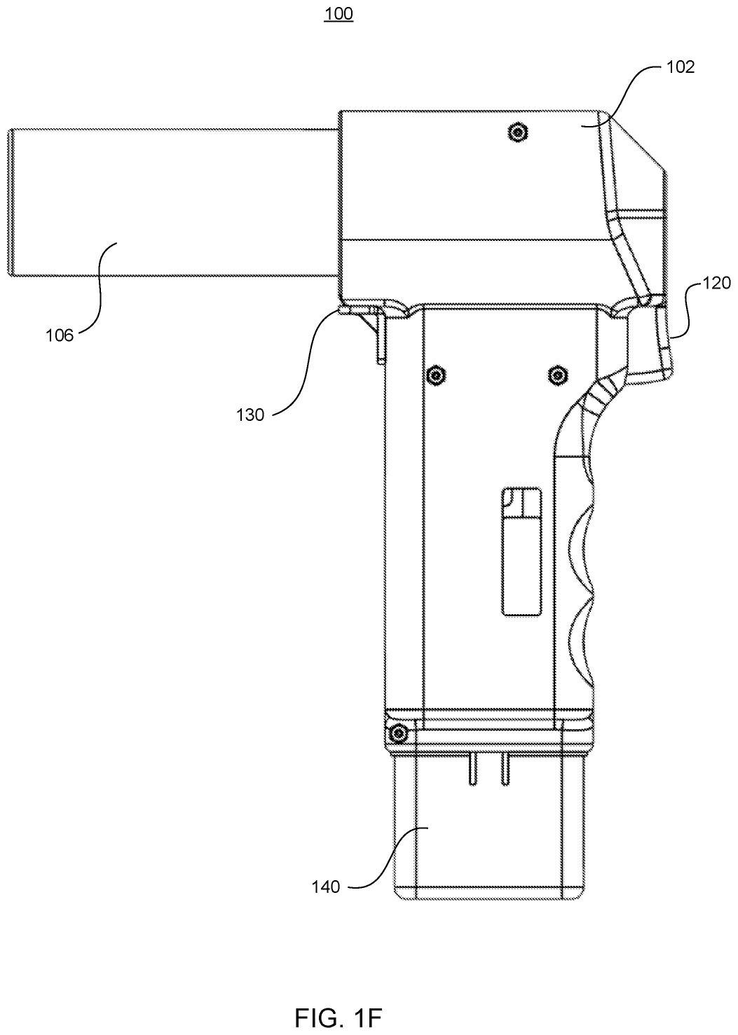

Now referring to the specific embodiments, FIGS. 1A-F illustrate various views of a gas-driven reciprocating mixing and injector system 100. FIG. lA is a perspective view of 100 illustrating the housing 102, the mixing activation mechanism housing 106, housing aperture 108, mixing trigger 120, venting lockout mechanism 130, and safety cap 140. These features can also be seen in the front and back views in FIGS. 1B-C, in the top and bottom views of FIGS. 1D-E, and the side view of 100 in FIG. 1F.

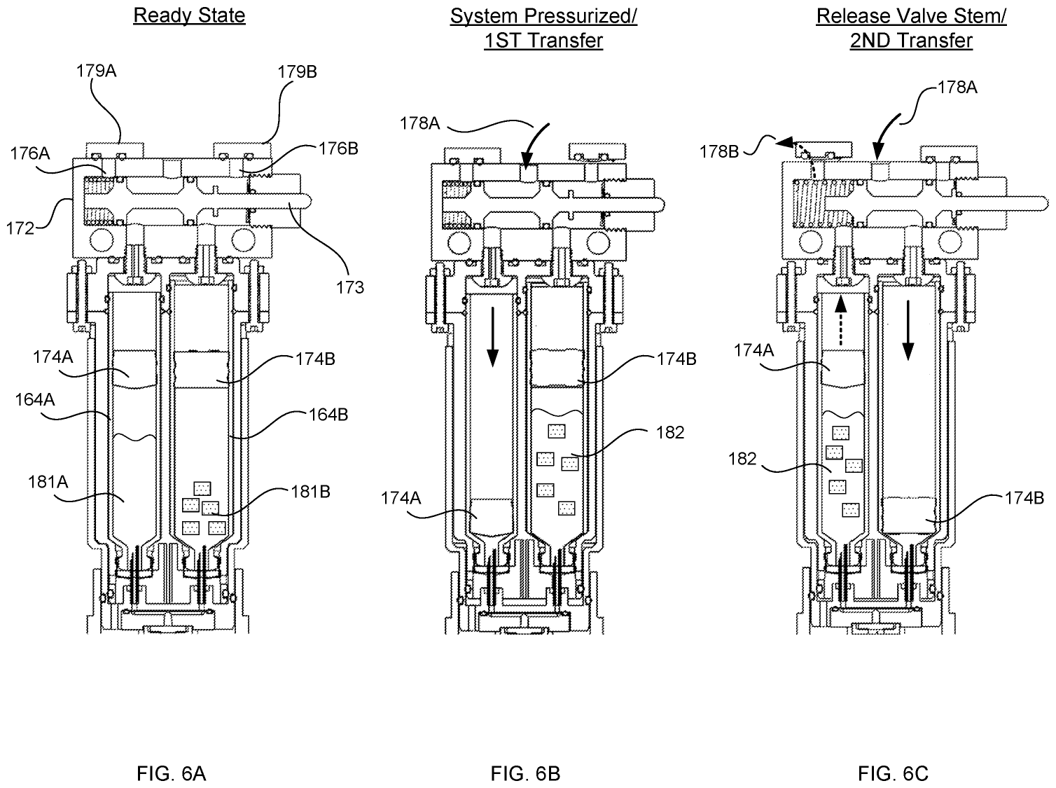

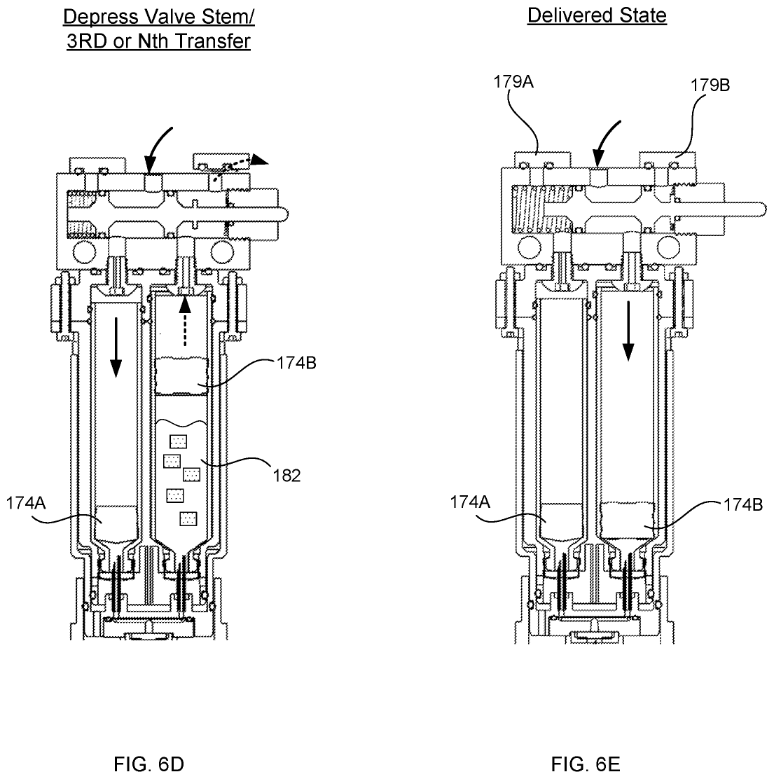

Additional components of the gas-driven reciprocating mixing and injector system 100 are illustrated in the cross-sectional view shown in FIG. 1G. A pressurized gas chamber 110 is situated in a gas chamber housing 111 and is initially separated from a gas piercing and gas/fluid communication member 112, which upon piercing provides gas/fluid communication to a gas regulator 113, which is configured to control the amount the pressure of the gas exiting the regulator 113 into the mixing system 170. A multi-directional valve 172 is part of the mixing system 170, which is configured to receive the controlled pressurized gas and redirect according to the positioning of the multi-directional valve. Further detail of the valve 172 will be provided in more detail below. The valve 172 interfaces with two gas-driven plungers 174A-B that are disposed within first and second containers 164A-B, each containing a first or second medicament component 181A-B (shown in FIG. 6A), which are initially separated from each other during the stowed state of the system 100.

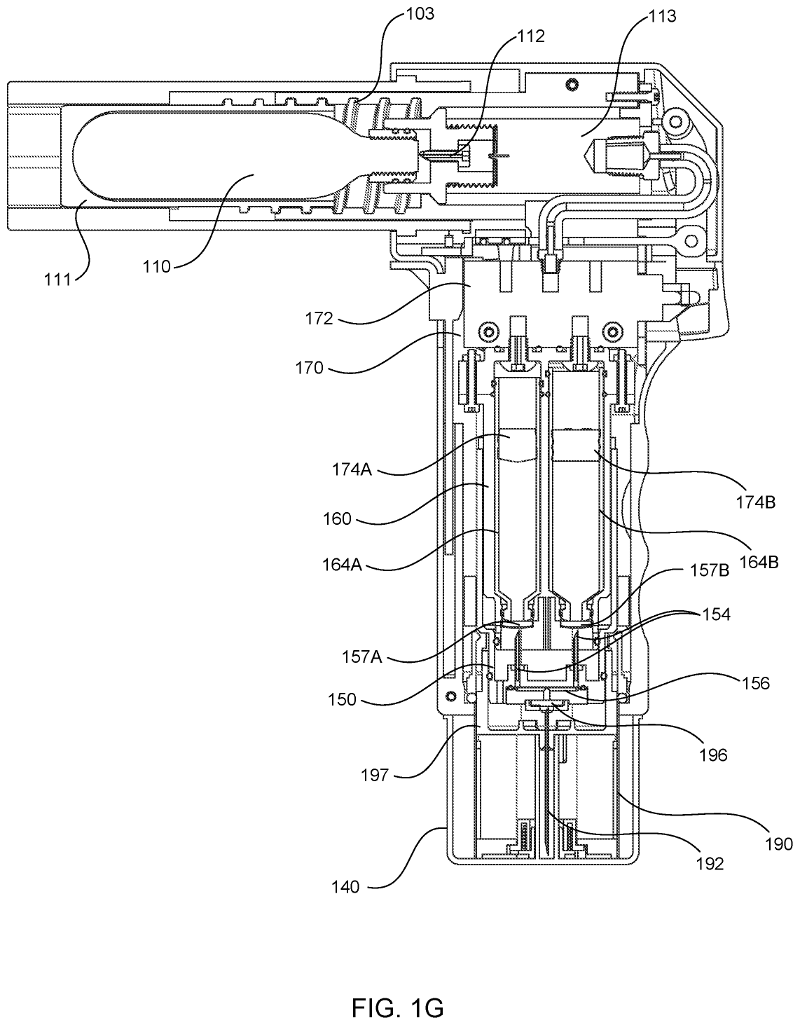

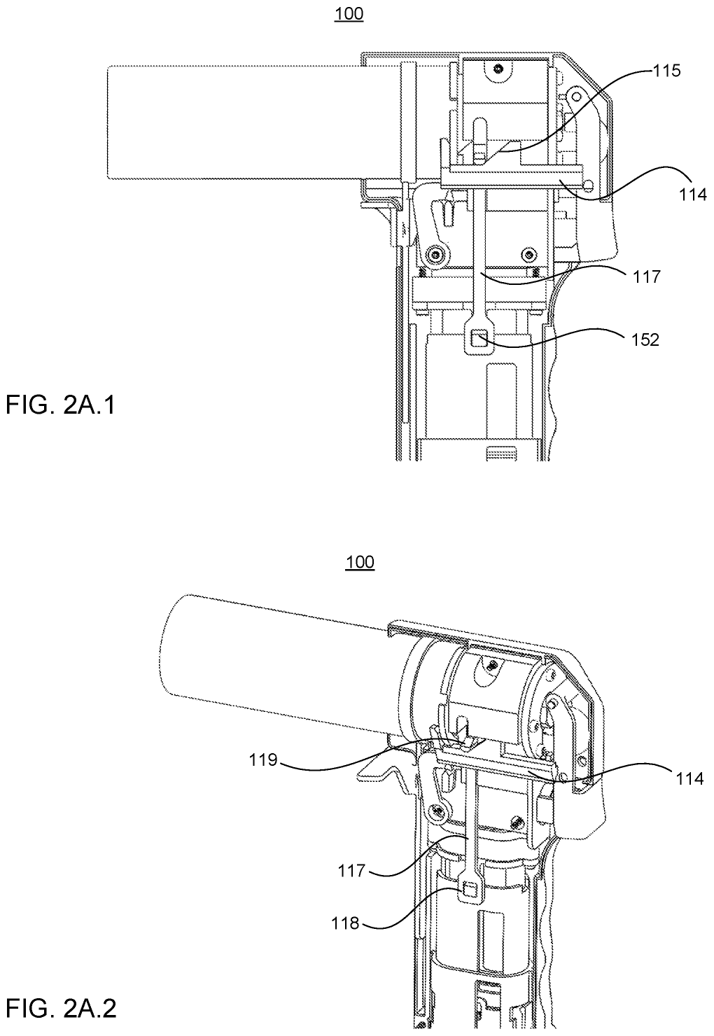

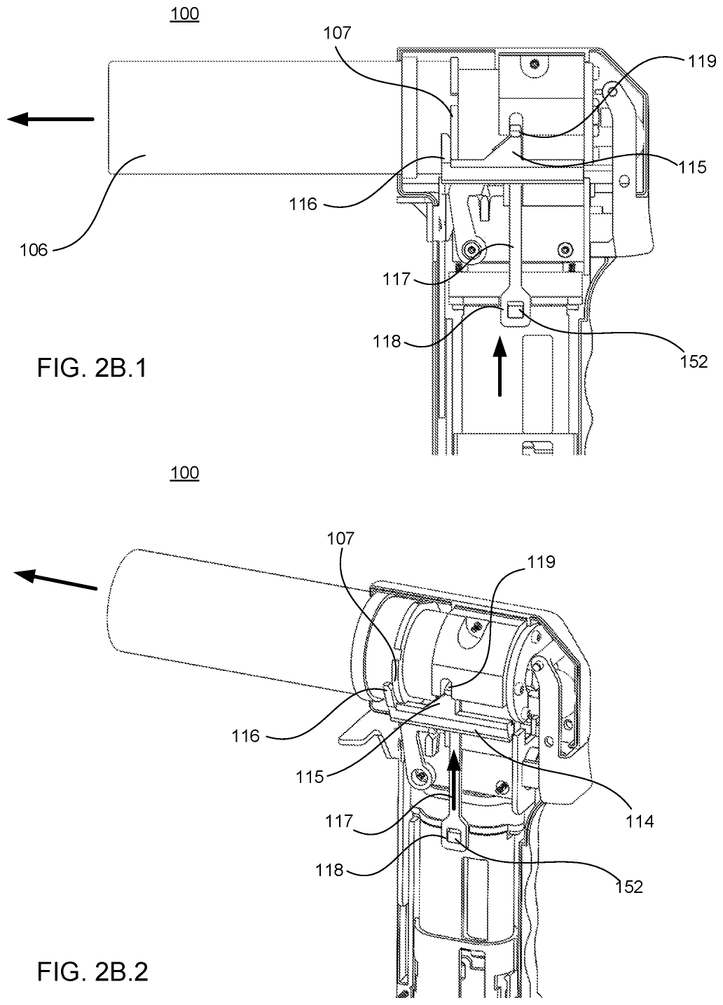

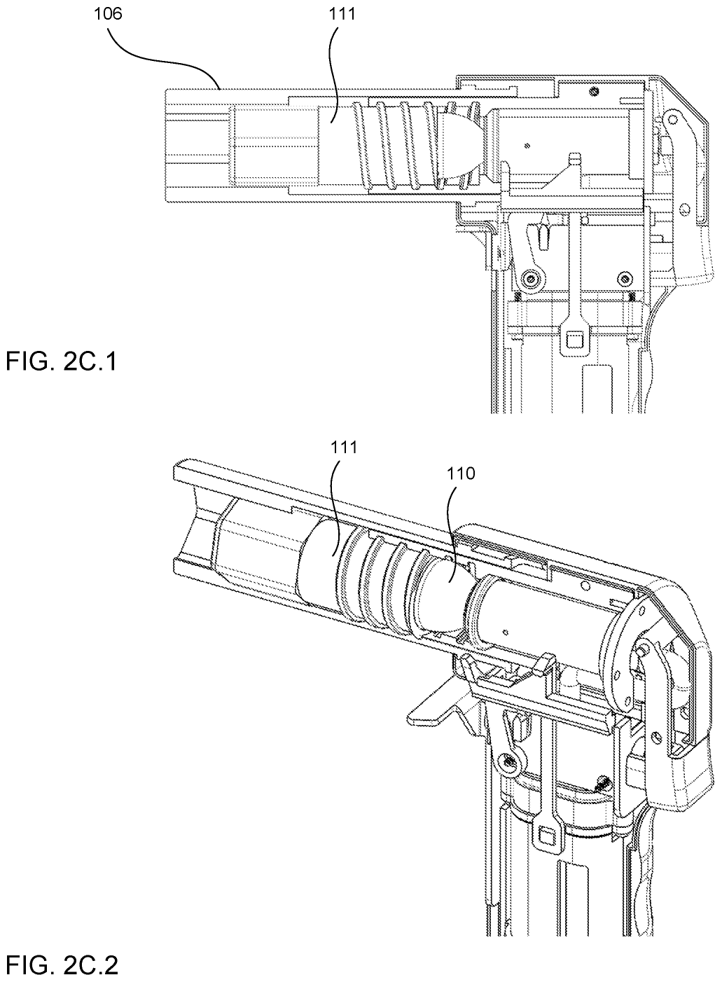

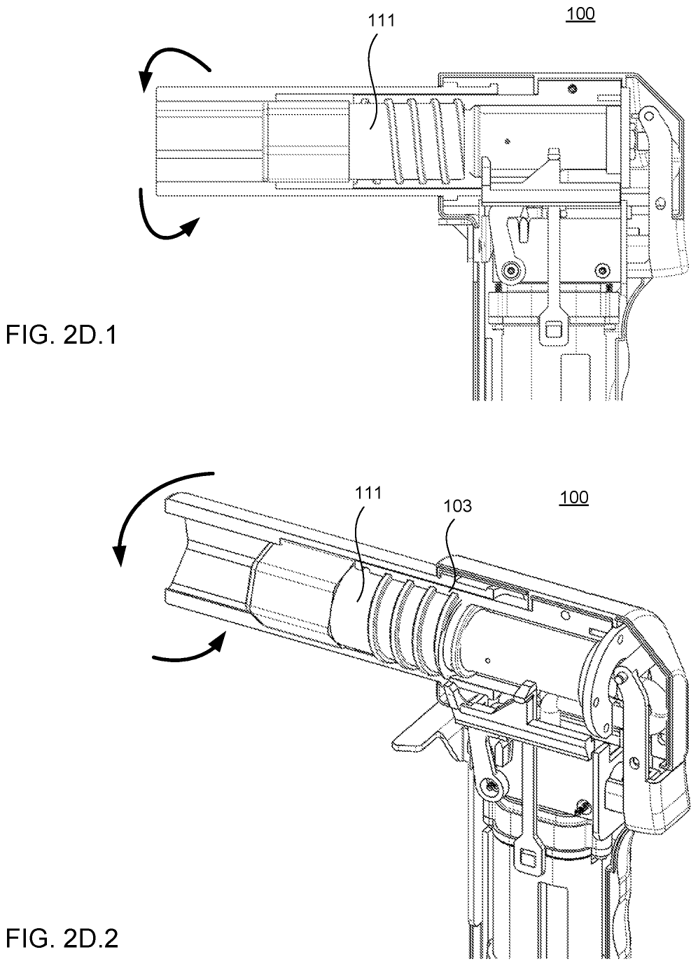

A fluid communication assembly 150 is positioned proximally to the containers 164A-B. It should be noted the first and second containers can be disposed within a cartridge container frame or housing 160. The fluid communication assembly 150 is comprised of a pair of mixing needles 154, a fluid communication channel 156, a frame (not labeled), and in this particular embodiment a fluid communication assembly tab 152 (illustrated in FIG. 2A.1).

The safety cap 140 covers the needle shield assembly 190, as well as the delivery needle 192.

FIGS. 2A.1-2B.2 illustrate various exposed views of the gas-driven reciprocating mixing and injector system 100 demonstrating engaging the fluid communication assembly 150 using the mixing activation mechanism, which includes the mixing activation mechanism housing 106. The mixing activation mechanism housing 106 is in mechanical communication with a mixing activation slide 114, which includes a ramped protrusion 115 positioned above a sliding base. A mixing activation strap 117 has a strap flange portion 119 that interfaces with the base of the mixing activation slide 114, until it is drawn up the ramp 115 portion of 114, by sliding or pulling 114 linearly, such that ramp 115 engages flange 119. On the other end of strap 117 is a strap connection interface 118 that mechanically interfaces and attaches to the fluid communication system tab 152. As 117 moves upwards, or in a distal manner, it pulls on 152, which forces 150, and in particular the mixing needles 154, to engage with the first and second containers 164A-B, pierce the seals about each container, and create fluid communication between the two containers.

In order to linearly move the mixing activation slide 114, the mixing activation mechanism housing 106, which includes a mixing activation mechanism flange 107, which is configured to engage the mixing activation slide flange 116 of 114, can be pulled away from housing 102 by a user. This pulling causes 106 to pull 114, which then causes strap 117 to move distally causing 150 to create fluid communication. Arrows shown in FIGS. 2B.1-B.2 show the lateral movement, which leads to the upward or distal movement.

Continuing to the next phase, FIGS. 2C.1-2D.2 illustrate various exposed views of the gas-driven reciprocating mixing and injector system 100 demonstrating activating the gas chamber 110 using the mixing activation mechanism, which includes the mixing activation mechanism housing 106. Here instead linearly pulling 106, the user now rotates 106, which ultimately causes the gas chamber 110 to be pierced by the gas piercing and gas/fluid communication member 112, that creates gas/fluid communication with the regulator 113. As the user rotates 106, screws on the gas chamber housing 111 engage with the subframe 103 that is disposed within housing, and pushes the gas chamber 110 into 112. It should be noted that one end of the gas chamber housing is hexagonal shape that is keyed or fitted to a complementary internal hexagonal shaped sidewall of 106. Thus, the user is able to pull 106 linearly without engaging 111, until the user rotates 106. The complementary shapes of the gas chamber and the activation mechanism housing can be a variety of shapes, such as square, octagonal, pentagon, and so forth. The hexagonal shape should not be construed to be a limiting shape.

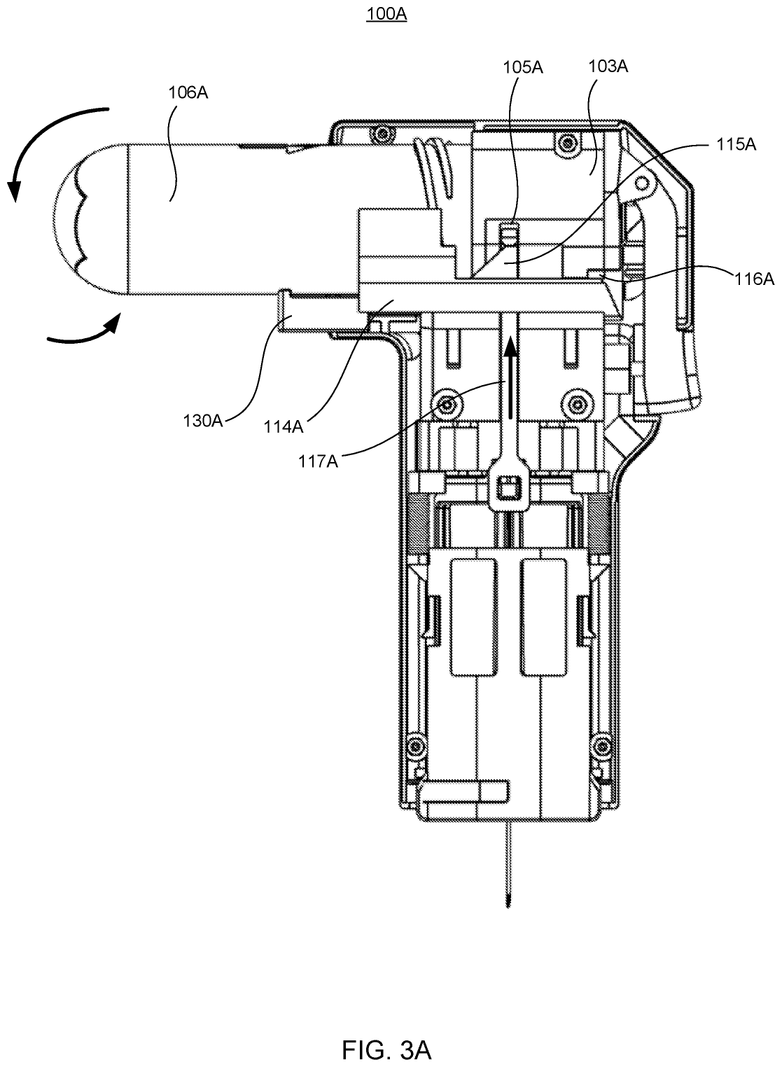

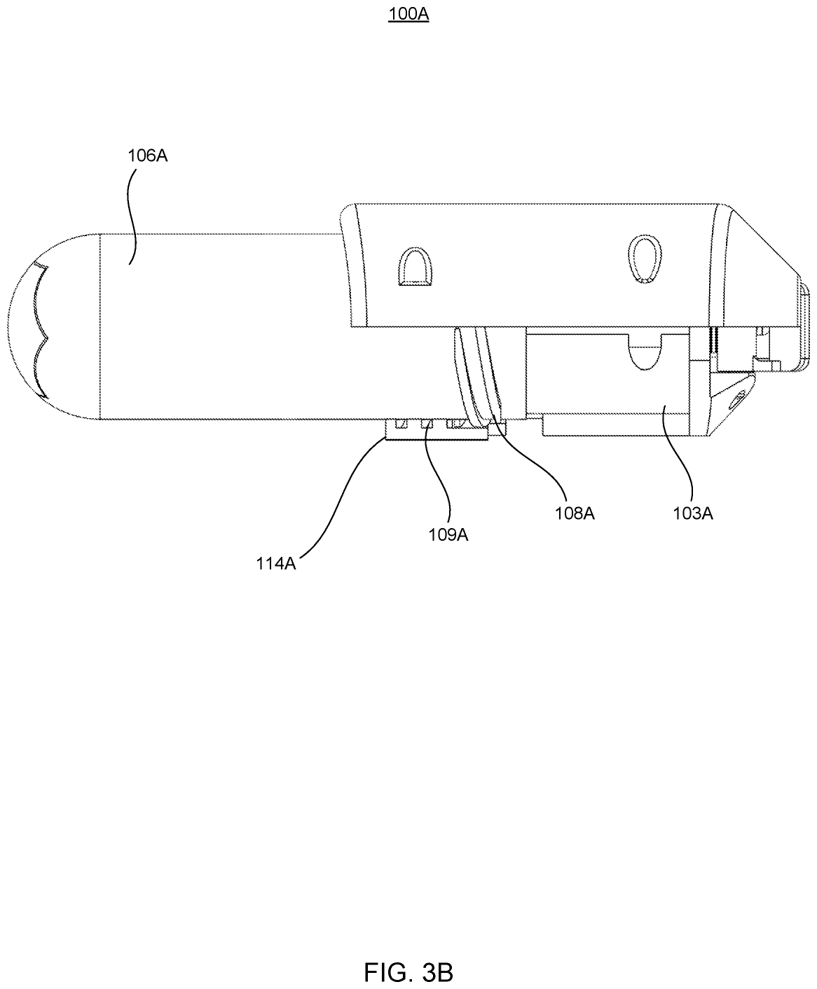

FIGS. 3A-B illustrate an alternative gas-driven reciprocating mixing and injector system embodiment 100A where the mixing activation mechanism, includes an alternative mixing activation mechanism housing 106A that is configured to provide multiple outputs using a single input or user motion. Here instead of pulling 106A in a linear manner, a user simply rotates 106A, which causes the two outcomes of fluid communication between containers 164A/B and mixing needles 154 while subsequently piercing the gas chamber. Similar to the above embodiment, 100A also includes a mixing activation slide 114A that interfaces with a mixing activation strap 117A in similar fashion, which as 114A is moved linearly, 117A is forced upwards or distally up the ramp 115A of 114A, which causes the fluid communication assembly 150 to engage the first and second containers and create fluid communication. To cause the linear motion of 114A, the user rotates 106A, which in this version includes threaded screws 104A that engage with screw channels 109A of 114A that are positioned on the back side or internal side of 114A, which is viewable from FIG. 3B. Once 117A is forced to the top of ramp 115A it becomes slotted in notch or channel 105A of subframe 103A, a flange 116A of 114A engages with a surface of 103A to prevent further linear motion. The user is allowed to continue rotating 106A, which continues to use the screw channels 109A of 114A to move 106A further into housing 102. A gas chamber 110A disposed within 106A, similar as to the above embodiment (but not shown), is then pressed into a piercing and gas/fluid communication member (again similar to 112 above, but not shown) until the gas chamber is pierced and fluid communication with the regulator of 100A occurs, which pressurizes the regulator and mixing system. The regulator of 100A is the same or similar to regulator 113 of system 100.

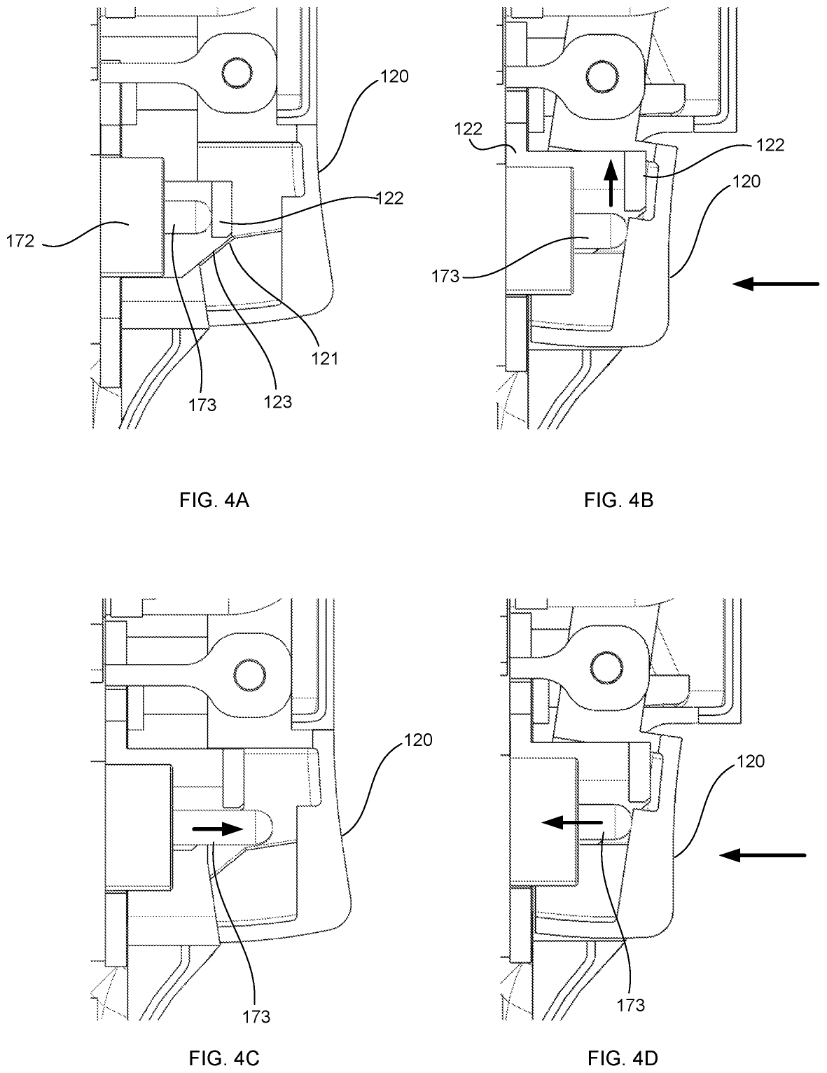

Once gas/fluid communication occurs and the regulator and mixing systems are pressurized, the user can now utilize the mixing trigger 120 to transfer fluid back and forth between the first and second containers. FIGS. 4A-D illustrate various phases of mixing trigger 120, which can be used with either system 100 or 100A. In the embodiment shown in FIGS. 4A-D, there is include a valve stem release slide 122. This release slide 122 initially keeps the valve stem 173 of valve 172 in a depressed state. When a user initial depresses mixing trigger 120, a mixing trigger angled interface 121 interfaces with a valve stem release slide angled interface 123 and forces release slide 122 upwards or in a distal manner, which then enables the valve stem to be released. Once the user releases pressure from mixing trigger 120, neither the release slide nor the mixing trigger are impeding valve stem 173 allowing it to protrude or extend outwards from the valve 172 as shown in FIG. 4C. When the user depressed the mixing trigger 120 again, it also depresses the valve stem 173, which as will be described further below changes the position of the internal pathways of the valve 172, thus redirecting pressurized gas received from the regulator and gas chamber in a particular manner. The valve stem 173 has a valve spring 171 that interfaces with it and forces the valve stem outwards, when it is not being depressed or otherwise impeded.

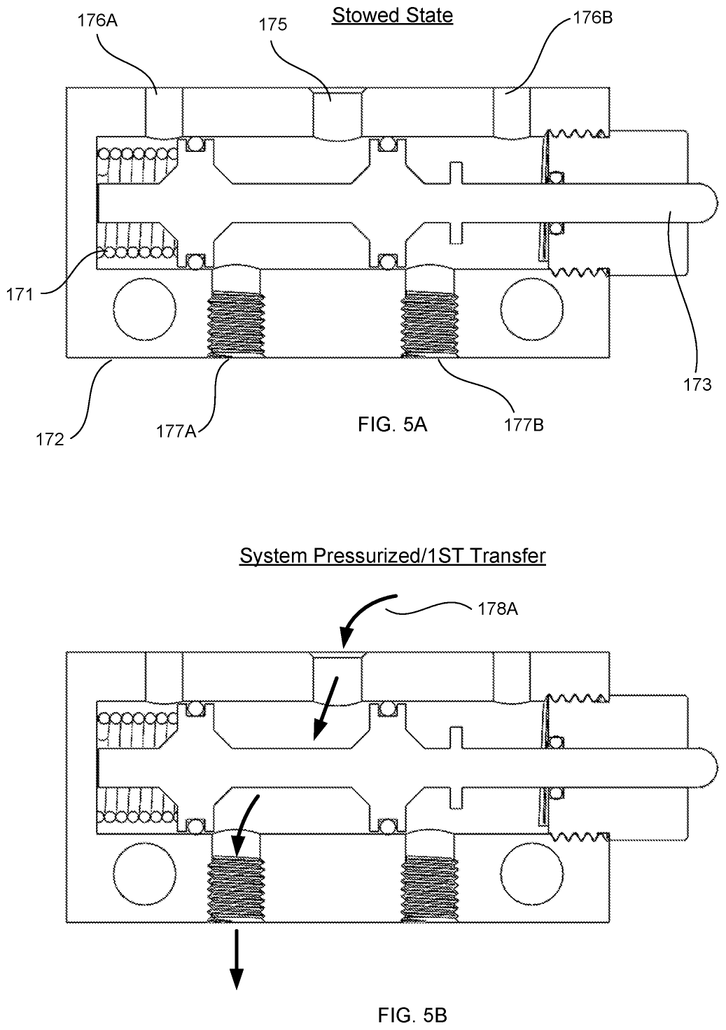

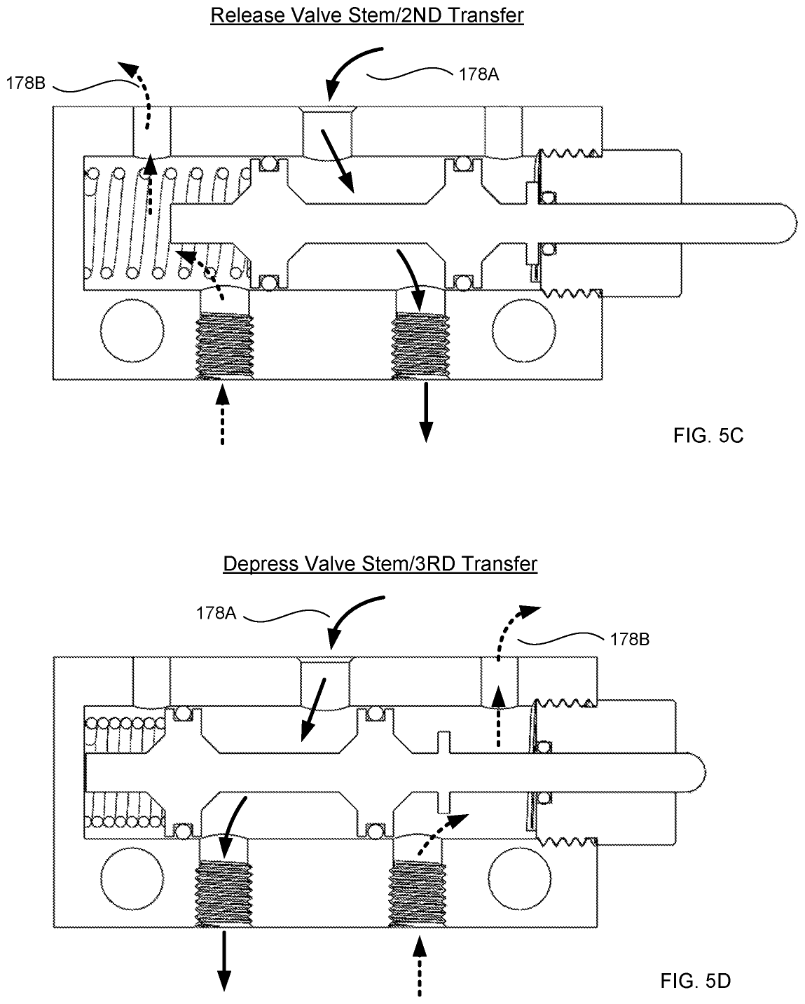

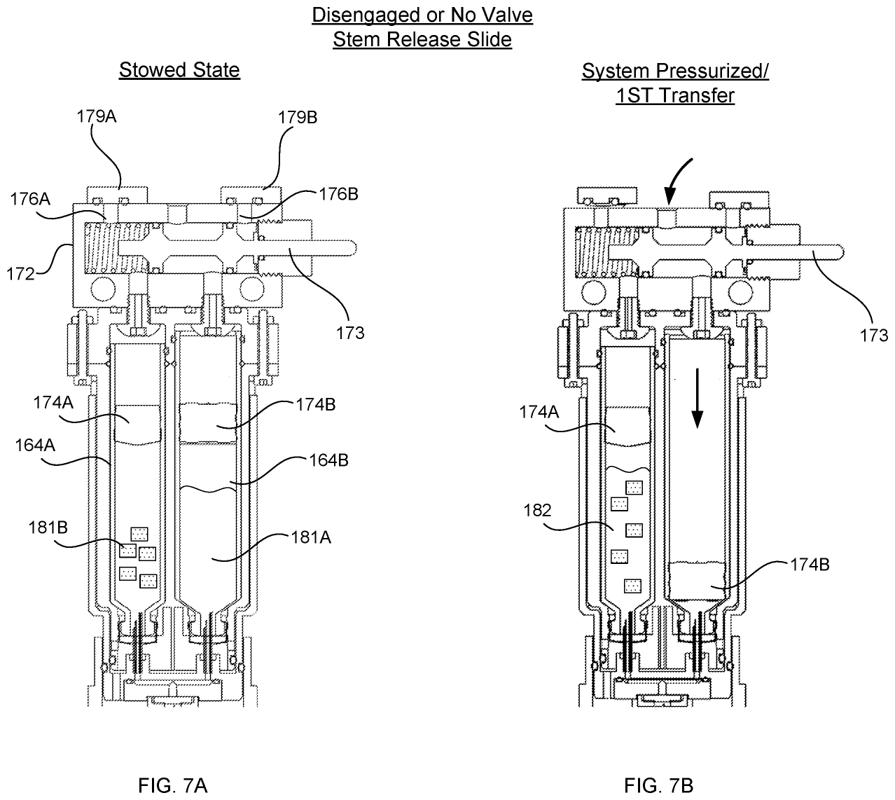

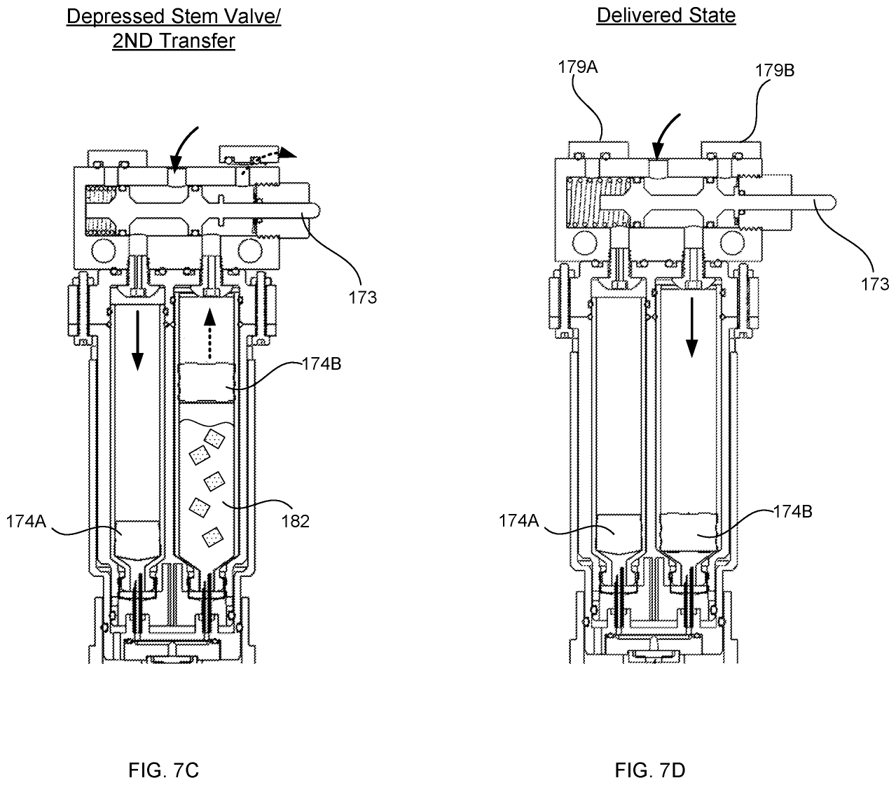

FIGS. 5A-D illustrate various phases of a multi-directional valve of a gas-driven reciprocating mixing and injector system 100 or 100A and the paths of pressurized gas. It should be understood that FIGS. 5A-D illustrate a valve 172 and phases based on the valve stem initially being in a blocked or depressed state. For example, if the system 100 or 100A included release slide 122. However, it should be noted that release slide 122 is optional, and the valve 172 could initially be in a position where the valve stem 173 is extended. This will be explored further in FIGS. 7A-D.

Referring now to FIG. 5A, where valve 172 is in a stowed state. There is no pressurized gas being directed into the valve via valve inlet 175 or directed out of gas plunger egress 177A or 177B. Pressurized gas is also not being directed out of venting ports 176A or 176B. When the system 100 or 100a is initially pressurized by piercing the gas chamber, the regulator sends pressurized gas into the valve inlet 175. The incoming path of gas 178A initially comes in through 175 and out of 177A to drive plunger 174A downwards. This initial pressurization also causes an initial transfer of the first medicament component 181A in the first container 164A to be transferred via 150 into the second container 164B, where it begins mixing with the second medicament component 181B to form a mixed medicament component 182. Once the mixing trigger 120, and thus the valve stem 173, is released, the incoming path of gas 178A is altered to where pressurized gas is now directed into 175 and out through 177B to drive the second plunger 174B of the second container 164B. The mixed medicament 182, currently in the second container, is now driven out of the second container and into the first container, where it pushes upwards or distally on the first plunger 174A. Gas that originally was pressing down on the first plunger is able to escape through vent port 176A as shown by the outgoing path of gas 178B shown in FIG. 5C. The mixing trigger 120, and thus the valve stem 173, can be depressed again, which alters the incoming gas path 178A to push down on the first plunger, which causes the mixing medicament 182 currently in the first container to transfer to the second container, force the second plunger upwards, where gas previously driving the second plunger downwards is now vented through vent port 176B as shown by the redirected outgoing path of gas 178B in FIG. 5D. At this point, those skilled in the art, can readily ascertain that the user can continue to depress and release the mixing trigger 120, which depresses and releases the valve stem 173, which alternates the gas paths going in and out, thus forcing the first and second plungers to be driven downwards (proximally) or upwards (distally) that in turn transfers the mixed medicament back and forth between the first and second containers as many times as the user decides. Each transfer back and forth helps to further mix or blend the medicaments components together as noted above, which is one of the problems to be solved because certain medicament components require additional or extra mixing energy to achieve a high-quality mixed medicament. In many cases, the mixing time itself can be reduced when compared to mixing that is achieved by simple shaking or swirling of the combined medicament components. It can readily be seen that using one of the systems of 100 or 100A, a user could easily depress the mixing trigger, 10, 20, 30, 40 or more times resulting in 20, 40, 60, 80 or more transfers between the first and second containers. The user can then check through housing aperture 108 to confirm the medicament looks fully mixed. An additional benefit of 100 and 100A is that the mixing can be deterministic and directly connected to the number of mixing cycles as a determination of completeness, rather than subjective determinations of mixing completeness, like visual inspection alone.

FIGS. 6A-E illustrate and provide additional clarity to the various states of a gas-driven reciprocating mixing and injector system 100 or 100A and the transferring of the medicament components between containers. FIGS. 6A-E show the interface between the valve 172, first and second containers 164A-B, and the fluid communication assembly 150. FIG. 6A illustrates a stowed state where no pressurized gas is acting on either of the first or second plungers 174A-B. Once pressurized, as shown in FIG. 6B, the first plunger 174A is driven into the first container and medicament component 181A is combined with medicament component 181B in the second container 164B to form mixed medicament 182. The valve stem 173 is release and the mixed medicament 182 is transferred from container 164B into 164A. The incoming and outgoing paths of gas 178A-B alternate as the valve stem 173 causes the alteration within valve 172. In FIG. 6D the user depresses the mixing trigger 120, which depresses the valve stem 173 and the transfer from first container to container of 183 occurs. Once a user is satisfied that mixed medicament 182 is fully mixed or homogenized, they can then proceed to the next phase, which is to lock out one or more venting ports (176A and/or 176B). Further details on how this is done are provided below. Once one or more ports art blocked and the delivery needle assembly is in fluid communication with the fluid communication assembly 150, the next release (as in this case) as shown in FIG. 6E causes the pressurized gas to force the mixed medicament out of the second container through the delivery needle into a recipient. It should be noted, that the system can prepared for the delivery state with either a release or a depression of the mixing trigger 120, prior to engaging the venting lockout mechanism, once the user is satisfied the drug product is properly mixed.

FIGS. 7A-D illustrate an alternative variation of the various states of a gas-driven reciprocating mixing and injector system and the transferring of the medicament components between containers where the valve stem 173 of the multi-directional valve 172 has an alternative starting position. As shown in FIG. 7A, the valve stem 173 is extended during the stowed stated, thus when the system is pressurized, as in FIG. 7B, the system pressurization causes the first transfer of medicament 181A in container 164B to go into 164A and mix forming mixed medicament 182. The user's first action on the mixing trigger 120 is a depression, which causes a second transfer or in other words the mixed medicament 182 to transfer from container 164A into 164B. If venting port 179A were to be blocked at this moment, when the user releases the mixing trigger, and thus releasing the valve stem 173, the mixed medicament 182 would then travel out of the container 164B through the delivery needle into a recipient, so long as the delivery needle is in fluid communication with fluid communication assembly 150. If not, then the mixed medicament would remain in the container 164B (and not transfer into 164A) until the delivery needle comes in fluid communication with fluid communication assembly 150.

It should be noted that the mixed medicament's final position could be in either container (164A-B) and either a release or depression on the mixing trigger could release the mixed medicament. It should also be noted, that although medicament component 181A is shown as a liquid it could also be a dry component, and vice-versa where medicament component 181B shown as a dry component, could also be a liquid component. It is generally desirable to have the first medicament component being transferred to be liquid, but not an absolute requirement. It is possible for both medicament components to be liquids. One of the advantages of the systems already described are that medicament components with varying viscosities, miscibility, compactness of powders and so forth can still be readily combined in these systems and on demand as needed in fairly quick and consistent manner.

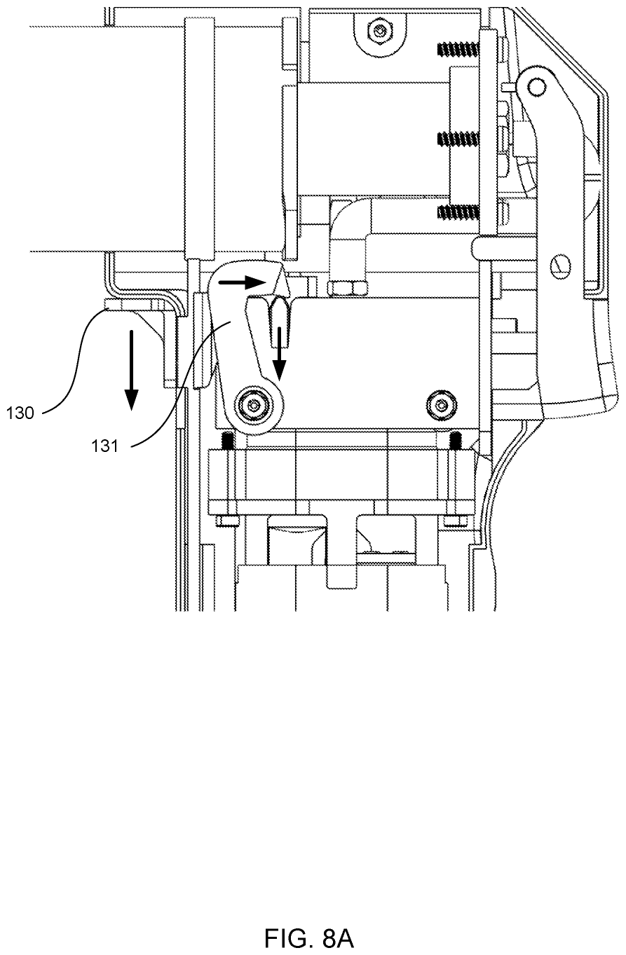

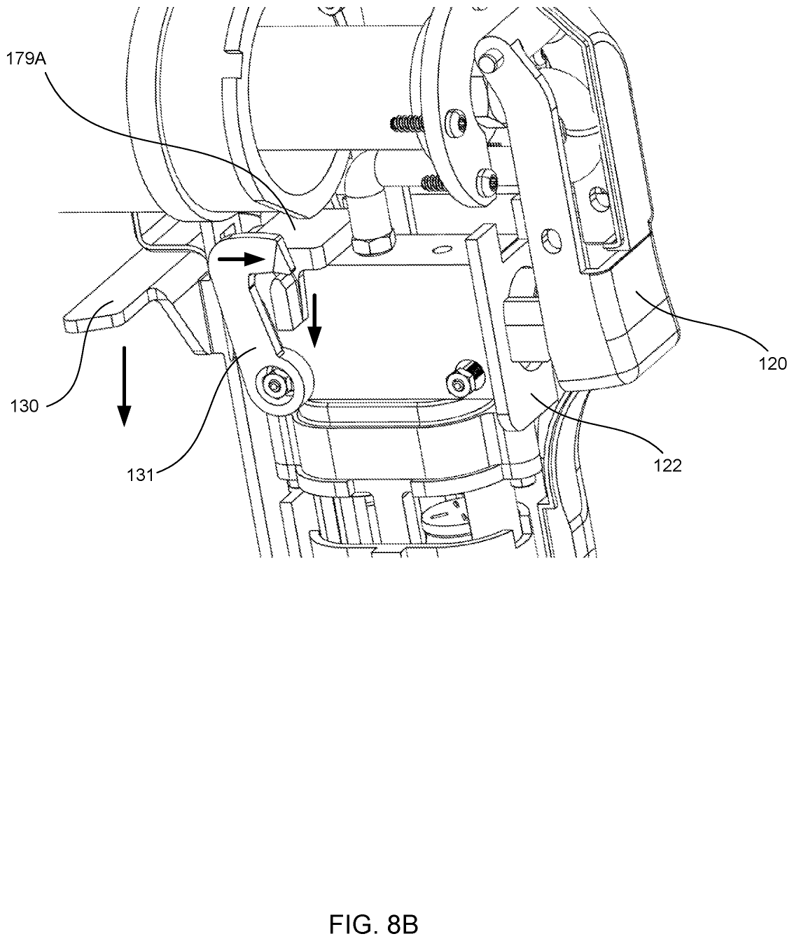

As just noted, it is important to block one or more of the venting ports of the valve 172 prior to delivering the mixed medicament. The embodiments shown in FIGS. 8A-B and 9A-B illustrate at least two versions of accomplishing the blocking of the venting ports. These are meant to be exemplary and not limiting, which is the intention of providing at least two examples. Now referring to the embodiment shown in FIGS. 8A-B, this is a vent obstruction method and system configured to block a single venting port, such as 176A. When the user depresses venting locking mechanism 130, it interfaces with a camming arm 131 that forces a vent obstruction component 179A down over (and in some cases into) venting port 176A. When using a single vent port obstruction system, it is important for the valve to be aligned in the appropriate position. In this case, the user would depress the mixing trigger 120 and while holding the mixing trigger 120 also depress venting locking mechanism 130. Thus, this ensures that the mixed medicament is leaving container 164B, such as shown in FIG. 6E and not returning to container 164A.

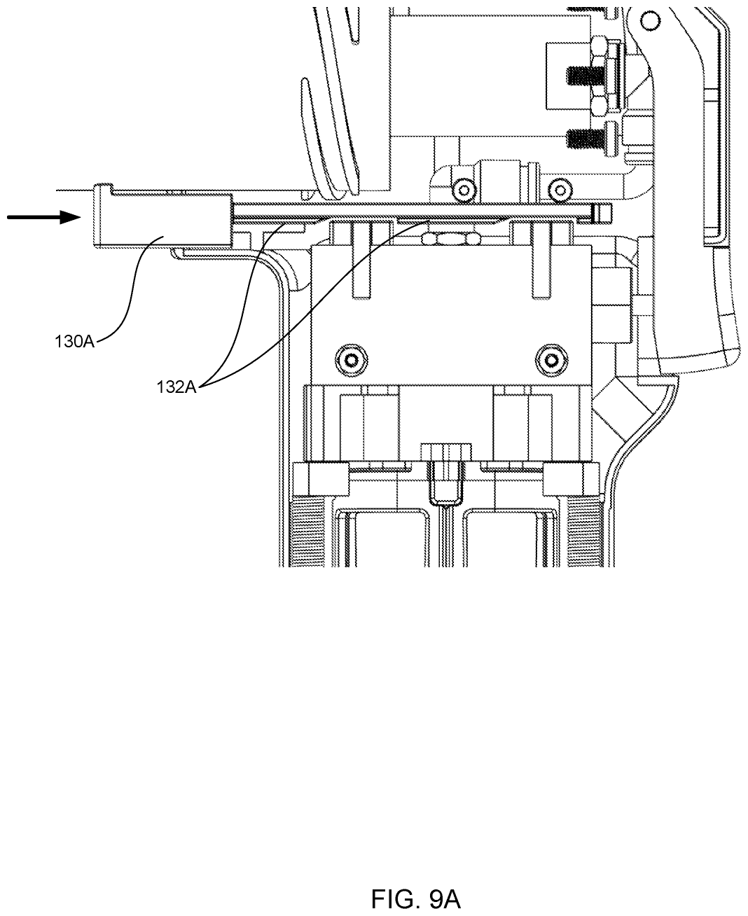

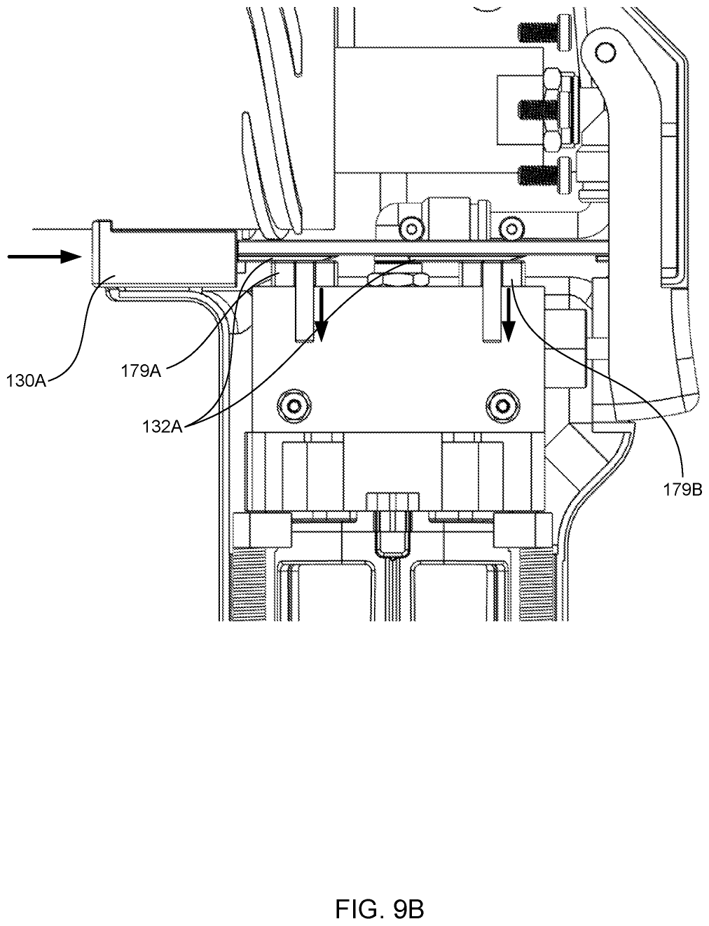

An alternative vent obstruction system is shown FIGS. 9A-B and is more agnostic to which container has the mixed medicament in it prior to delivery. This is because in this embodiment, venting lockout mechanism 130A is a sliding mechanism that has two ramped protrusions 132 that when slid over vent obstruction components 179A and 179B forces both of the vents 176A and 176B to be closed. In the variation shown, 130A is pushed into the housing, but it should readily be recognized that a version where the user pulls 130A out of the housing while accomplishing the purpose of both vents being obstructed.

In order to dispense the mixed medicament 182, the needle shield assembly 190 is depressed on the injection site, thus pushing the needle delivery assembly 197, along with the delivery needle 192, into the delivery septum 196 creating fluid communication between the delivery needle 192 and the fluid channel 156. The piercing of the delivery septum 196 causes the previously pressurized container containing mixed medicament 182 to be forced through delivery needle 192.

For additional context on how the needle shield assembly 190 and delivery needle 192 function and interface with the fluid communication system, the inventors refer to the published patent application noted above as well as some of the embodiments to be shown below. One of the primary focuses of the systems 100 and 100A is to convey an improved reciprocating mixing and injector system capable of mixing difficult to mix medicament components.

The remaining embodiments provided below include reciprocating mixing and injector systems that utilize various springs and mechanisms for the back-and-forth transfer of medicament components from one container to another container.

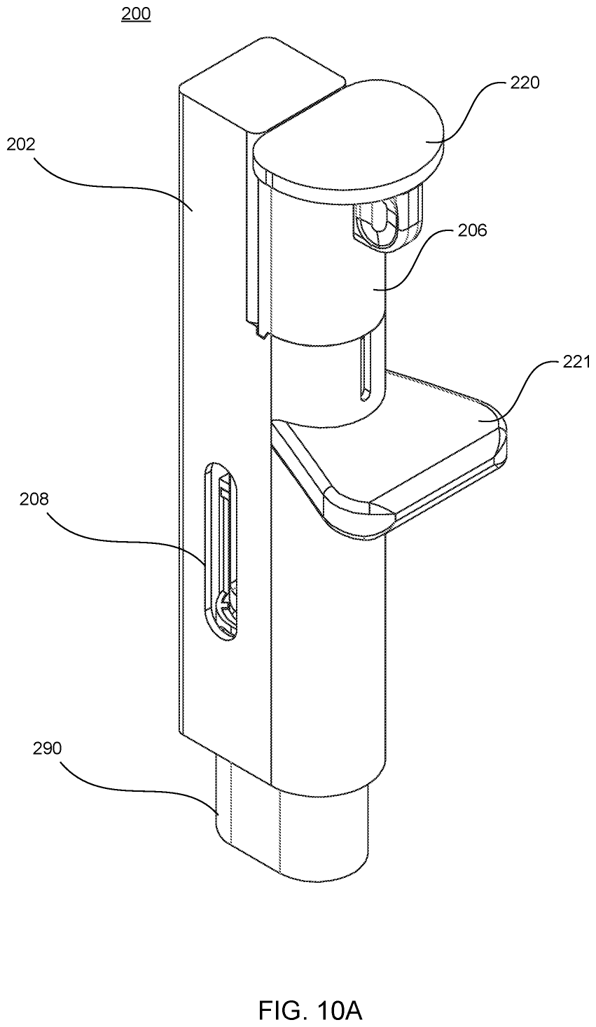

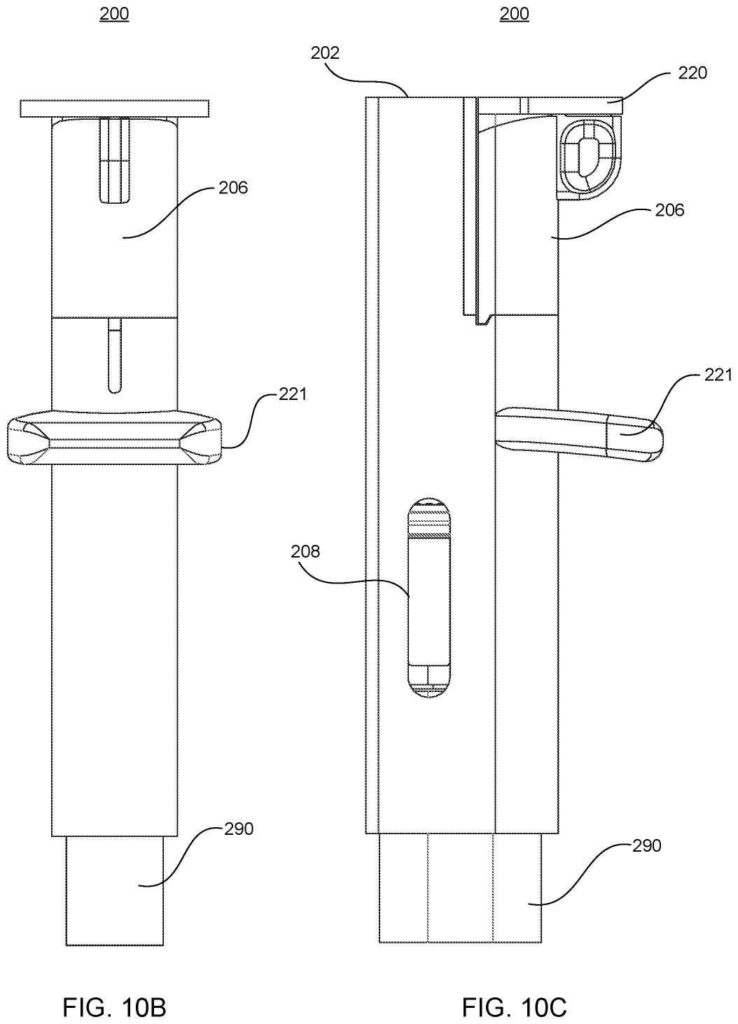

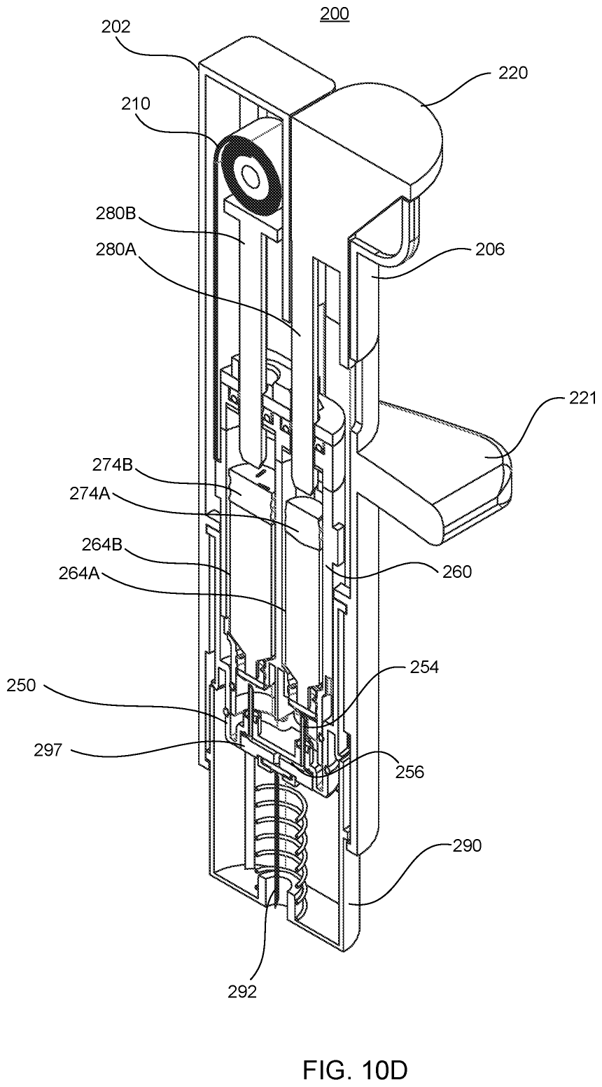

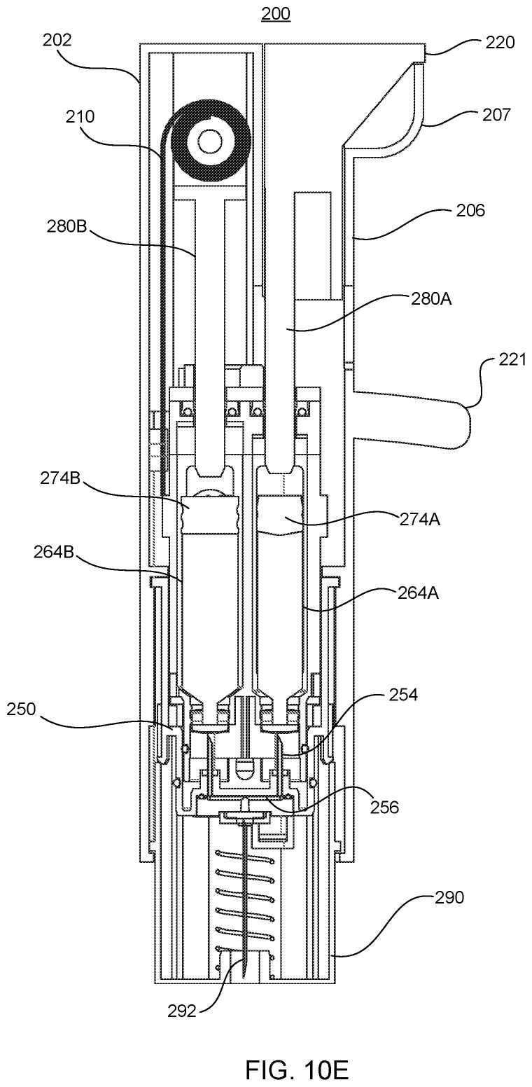

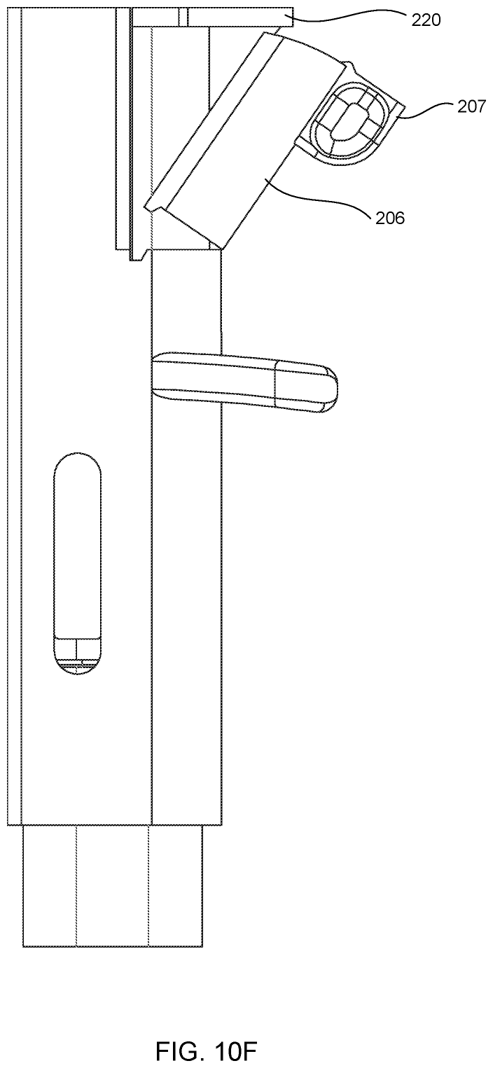

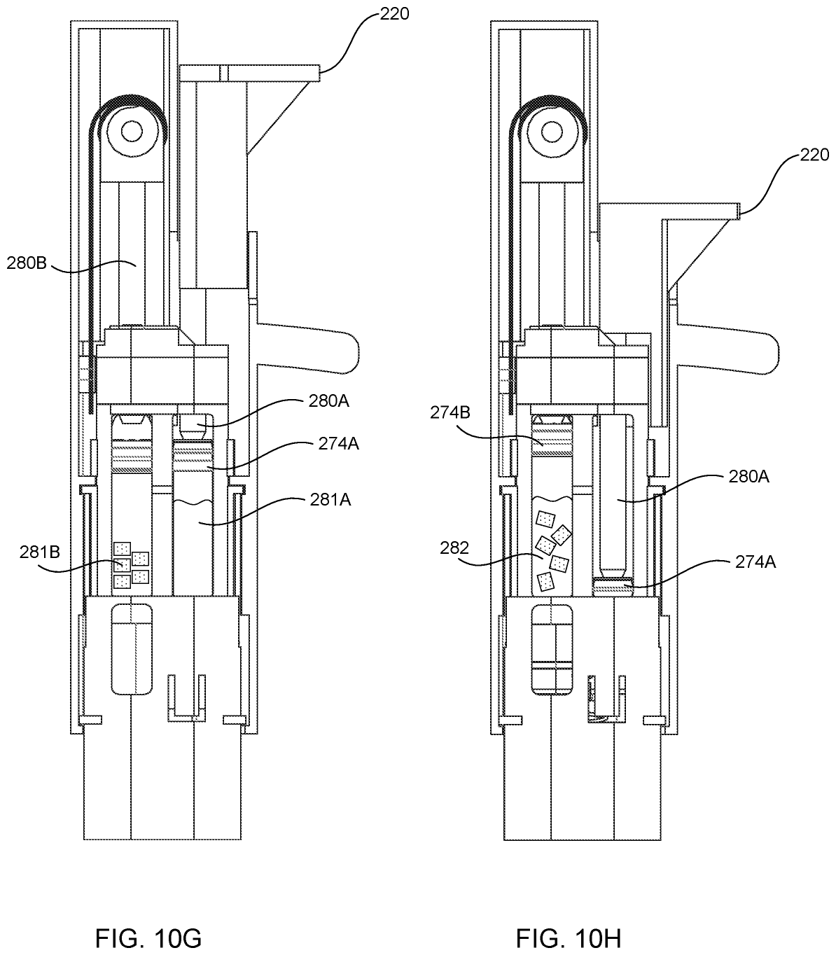

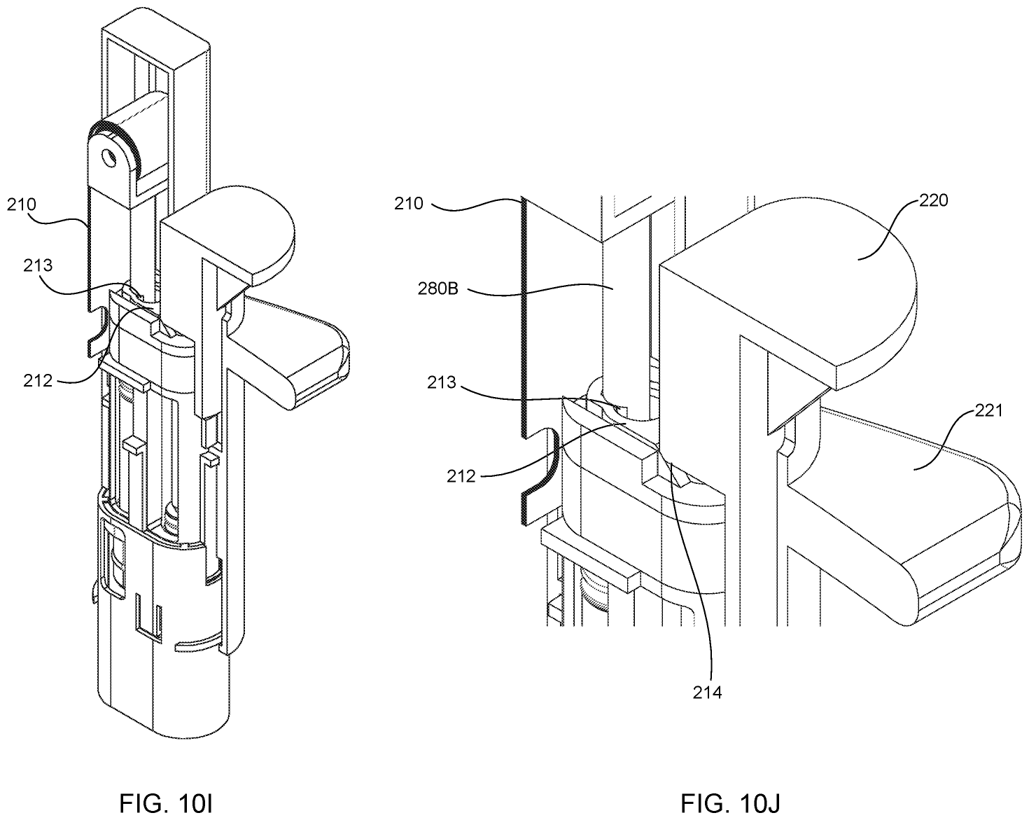

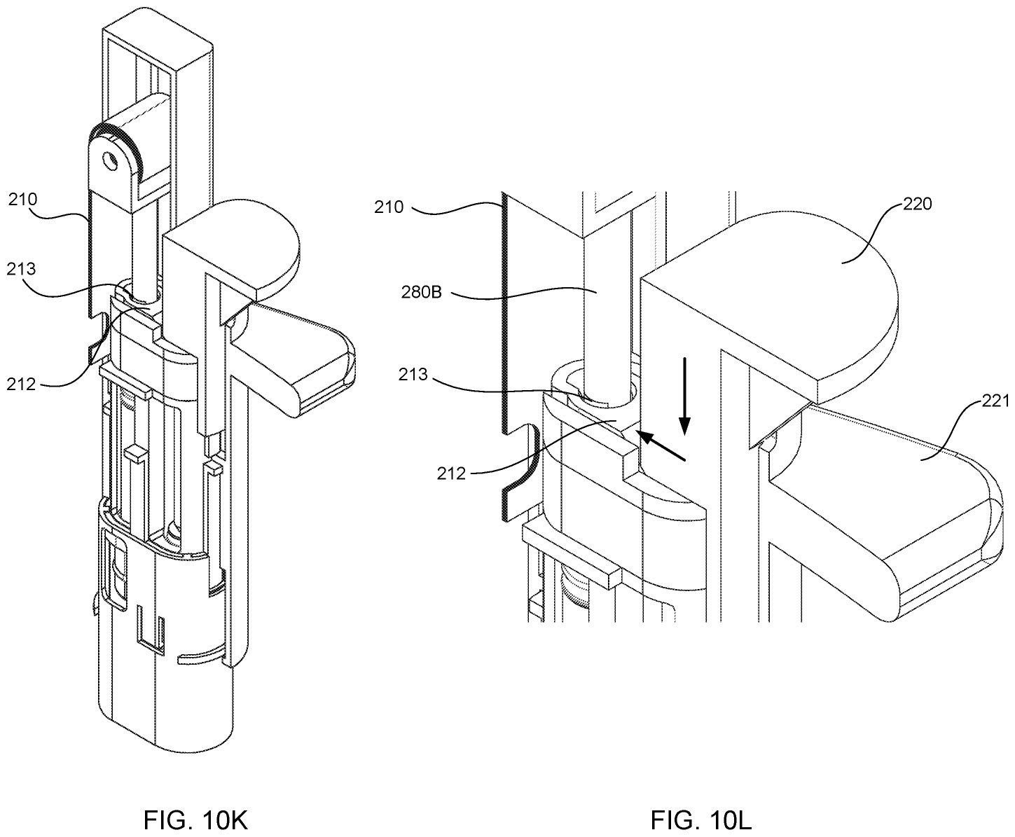

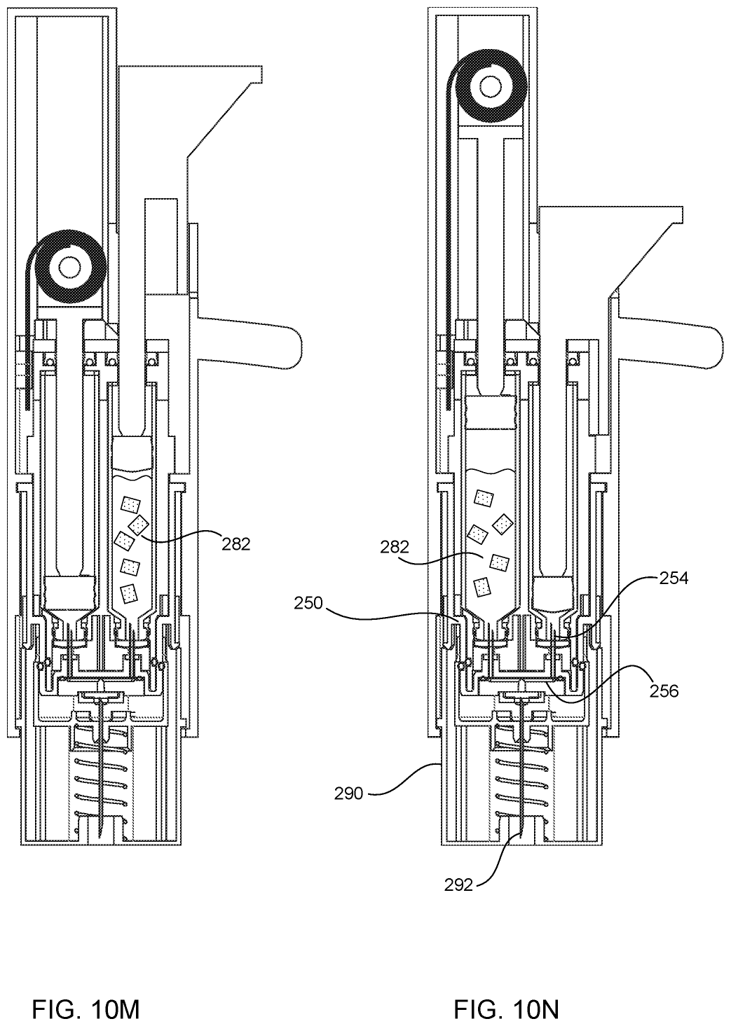

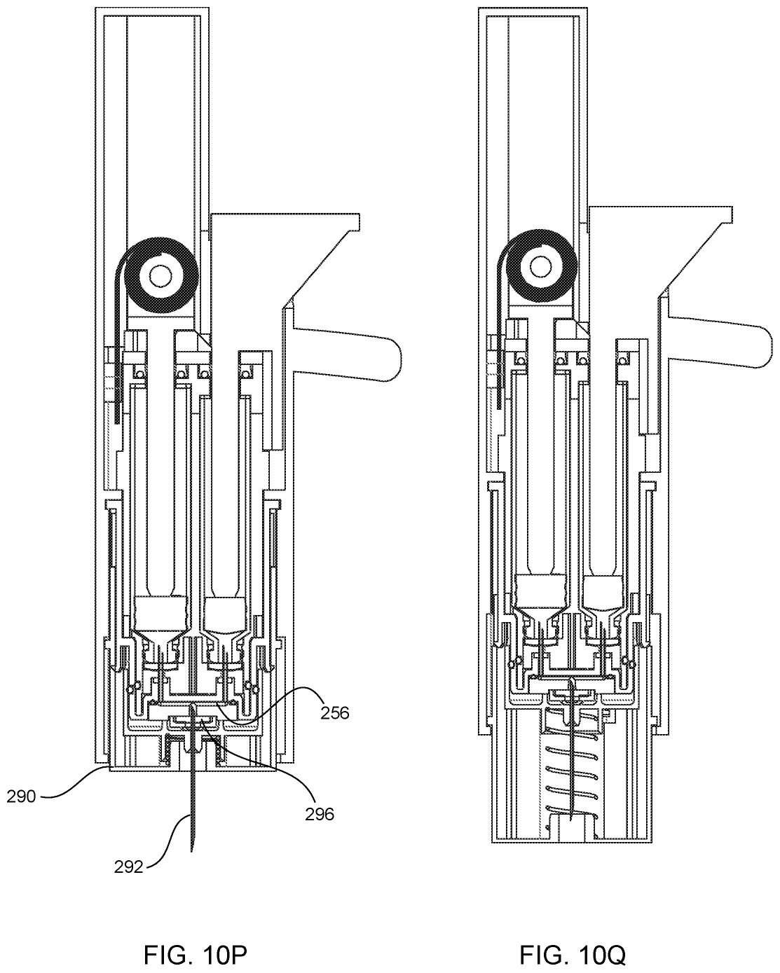

One such example is shown in FIGS. 10A-C which illustrate various views of an embodiment of an inline hand compression reciprocating mixing and injector system 200. System 200 is comprised of a housing 202, having an aperture 208, a safety/activation release 206, a needle shield assembly 290 and mixing grips 220 and 221, where mixing grip 220 is a movable mixing grip and 221 is a stationary or non-moving mixing grip.

FIGS. 10D-E illustrate cross-sectional views of the inline hand compression reciprocating mixing and injector system 200 of FIGS. 10A-C in a perspective cross-sectional view (10D) and side cross-sectional view (10E) to further illustrate the several components that enable this embodiment to store, mix and deliver a mixed medicament component 282 formed of first and second medicament components 281A-B. A constant force spring 210 is associated with a plunger rod 280B, whereas plunger rod 280A is directly coupled to mixing grip 220. Situated below each of the plunger rods are plungers 274A-B each disposed in one of containers 264A-B, each which hold a medicament component 181A-B. Similar to the gas-powered embodiments, containers 264A-B are held in place using a cartridge container frame 260, which can be driven into the fluid communication assembly 250 having mixing needles 254 and fluid communication channel 256. A needle shield assembly 290 is configure to be initially compressed during a delivery phase to expose the delivery needle 292, but then become fixed into place thereafter to prevent accidental future injuries from the sharp delivery needle 292.

FIG. 10F illustrates the removal of a safety/activation release 206 component of the inline hand compression reciprocating mixing and injector system 200, which allows the mixing grips 220 and 221 to be compressed together by a user's hand.

FIGS. 10G-H illustrate the phases of creating fluid communication between containers 264A-B and transferring a first medicament component 281A from one container to another to begin mixing with a second medicament component 281B to form a mixed medicament 282. When system 200 is in a stowed state there is no fluid communication between the containers. Once safety 206 is removed, the user can begin to compressing the mixing grips. Mixing grip 220, which is directly coupled to plunger rod 280A engages plunger 274A. As a result of medicament component 281A being a fluid, which most fluids are incompressible, the force is transferred to the cartridge container frame 260, which forces both containers onto the fluid communication assembly 250, where the mixing needles 254 pierce into each container and create a fluid communication pathway between each container. As the user continues to press further downward, the plunger rod 280A can now act further on plunger 274A to force the liquid medicament component 281A out of container 264A through 250 into container 264B.

FIGS. 10I-L illustrate various views of releasing the rod sliding lock 212 from a locking position to an unlocked position, so that the constant force spring 210 can act on driving plunger rod 280A downward or proximally. Another action that occurs when the user first compresses the mixing grips, is as the travel of the mixing grip goes far enough, a sliding lock engagement flange 214 of 220 interfaces with a ramped portion of rod sliding lock 212 and causes 212 to move in an orthogonal or lateral direction with respect to the up and down direction of the mixing grips and plunger rods. This lateral shifting releases a notched portion 213 on plunger 280B from engaging with a ledge of 212 and preventing plunger rod 280B from traveling downward. Once released, as the user begins to decompress or release their grip on the mixing grips 220 and 221, the constant force spring 210 can now drive the plunger rod 280B downward, which forces mixed medicament in 264B back into container 264A and forces the plunger rod 280A upwards.

The reciprocating back and forth transfer between containers and ultimate delivery of the mixed medicaments is further shown in FIGS. 10M-Q, which illustrate these various phases of from a ready-to-mixed phase to a delivered phase including the extension of a needle shield after the injection phase. FIG. 10M illustrates the phase where the rod sliding lock 212 has been released the constant force spring 210 has driven plunger rod 280B down, which has caused plunger rod 280A to go up as just noted. The mixed medicament component 282 is now in container 264A. The user can now compress mixing grips as many times as necessary, such as shown in FIG. 10N, to continuously transfer the mixed medicament 282 back and forth between containers until they are satisfied mixing of the medicament is sufficient, which can be supported by viewing the mixed medicament through the aperture 208 or referencing a predetermined number of counts and/or mixing cycles. With each release of the mixing grips the constant force spring 210 transfers the mixed medicament component back to container 264A.

When the user is ready to deliver the mixed medicament 282, the user while compressing the mixing grips, can depress the needle shield assembly 290 over the injection site, which upon being initially depressed, compresses and uncovers the delivery needle 292. When the delivery needle is further depressed or injected into a recipient, it causes the distal end of the delivery needle to pierce a delivery septum 296 that creates fluid communication with fluid communication assembly 250. Once the fluid communication is created, the constant force spring 210, which is continually acting on plunger rod 280B, can now drive the mixed medicament 282, which is now in container 264B, as a result of compressing the mixing grips, into the recipient through the delivery needle 292. When the user pulls the needle out of the recipient, a spring in the needle shield assembly causes it to extend and lock in to place as shown in FIG. 10Q.

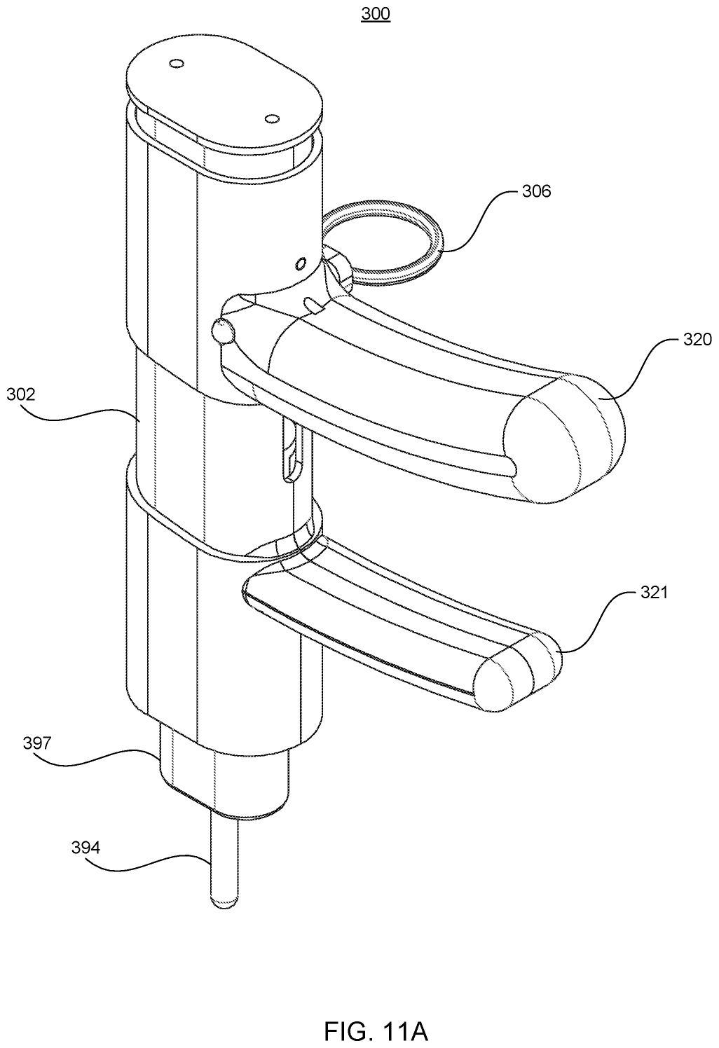

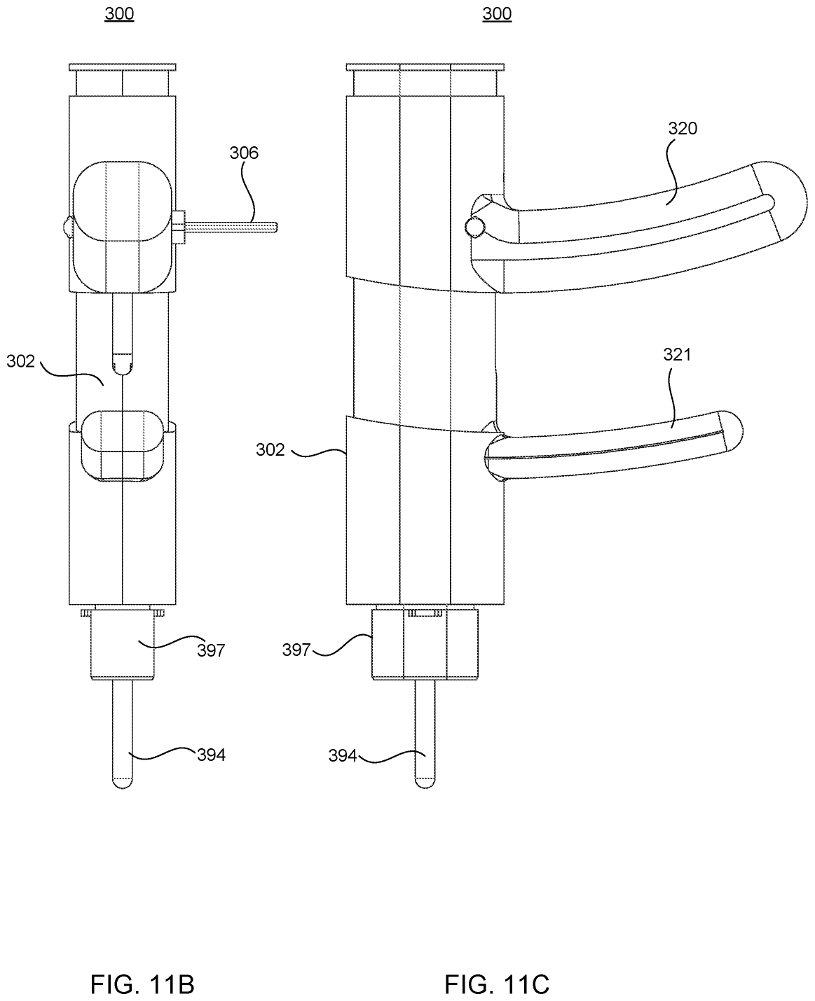

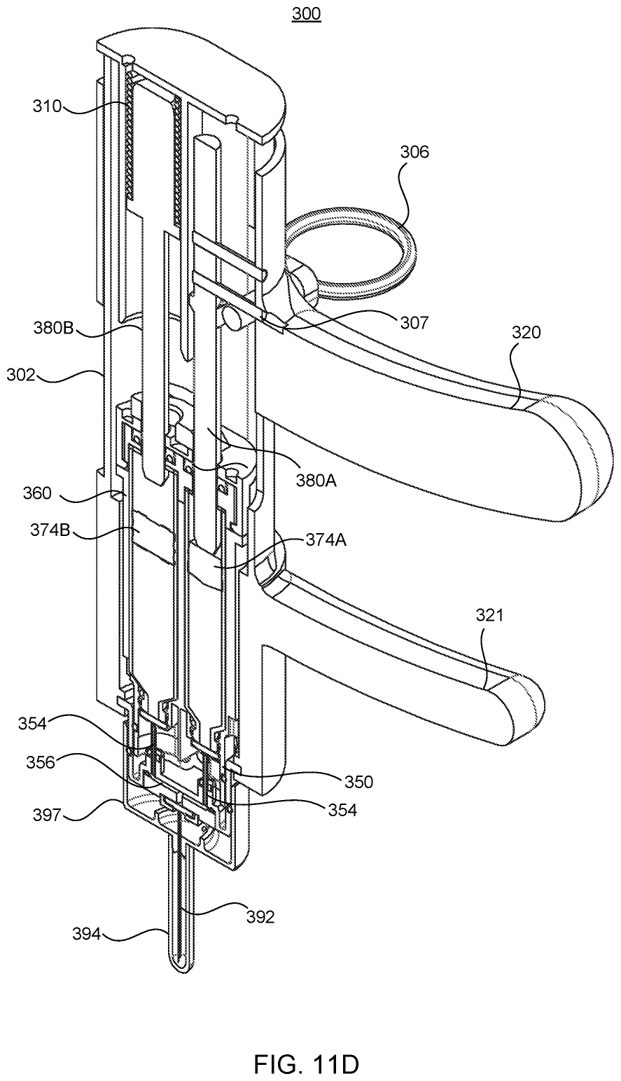

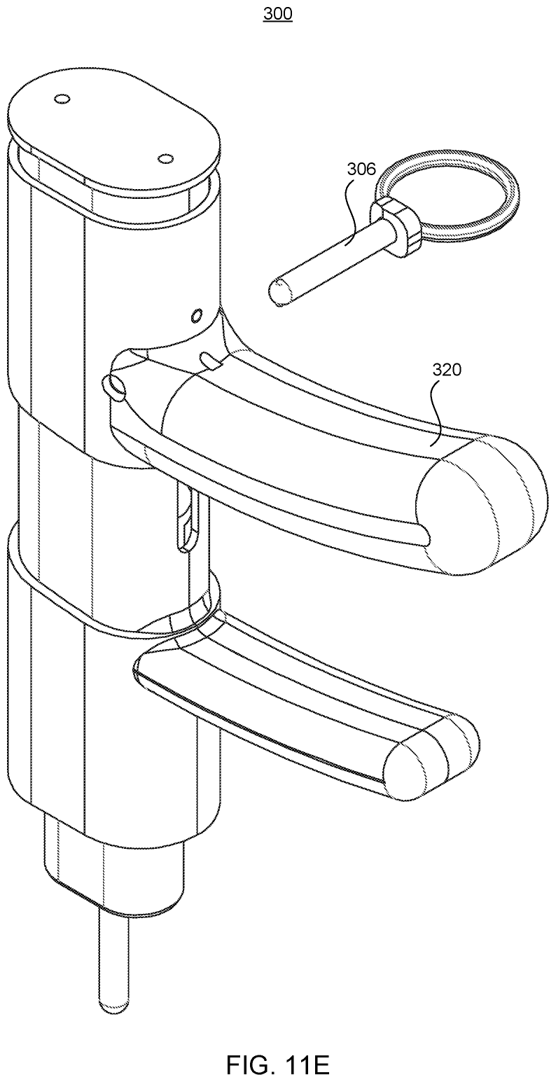

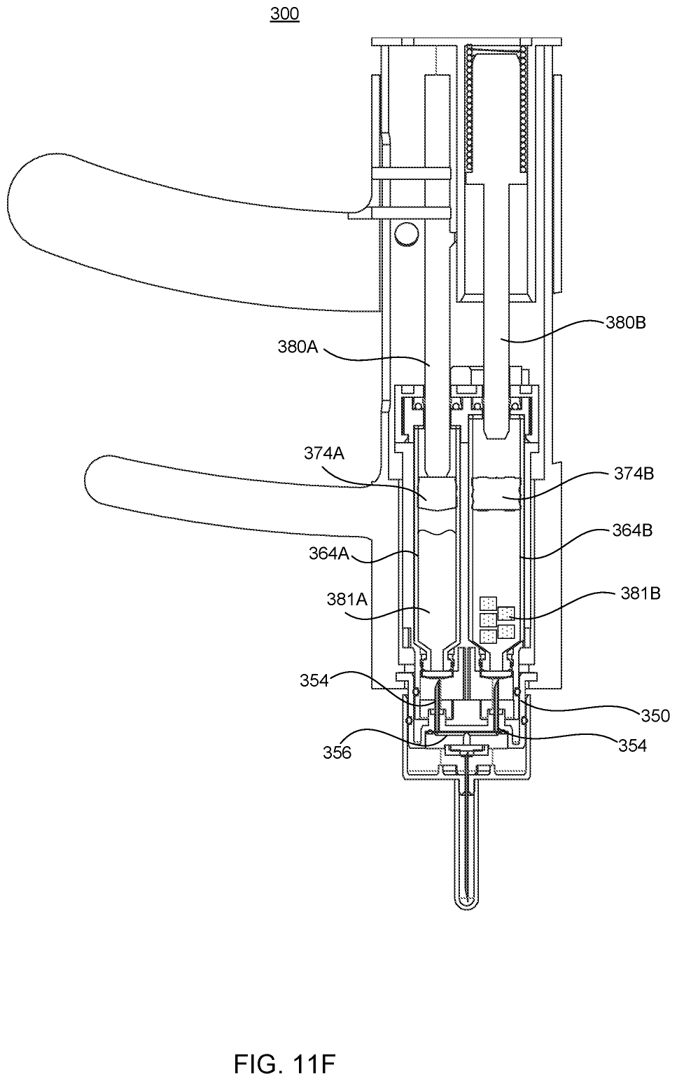

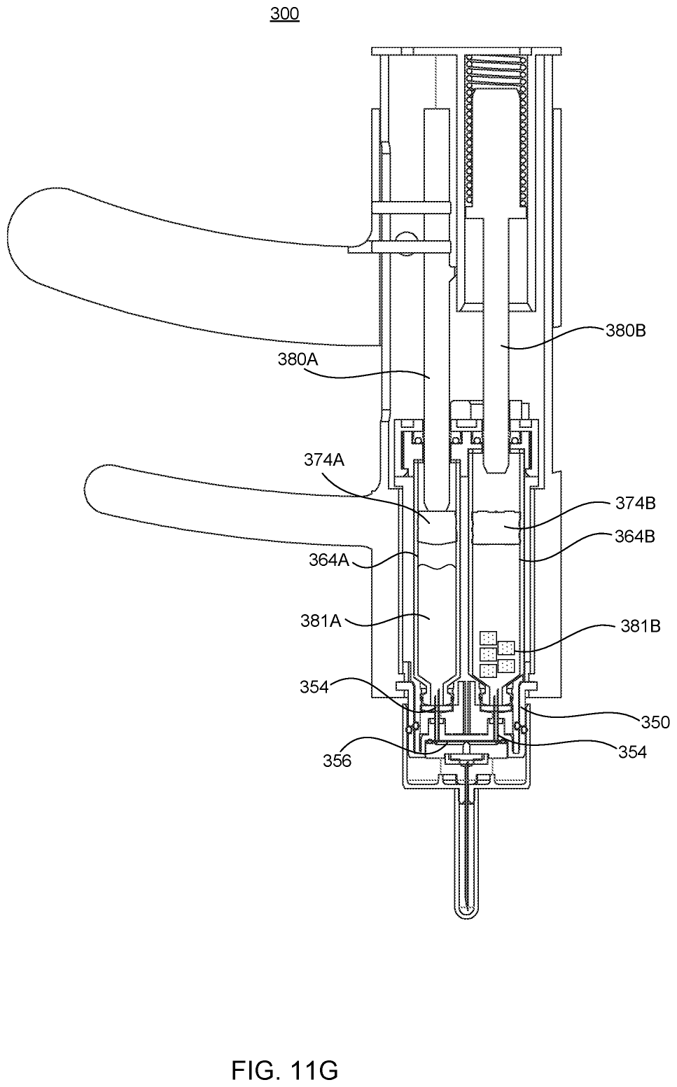

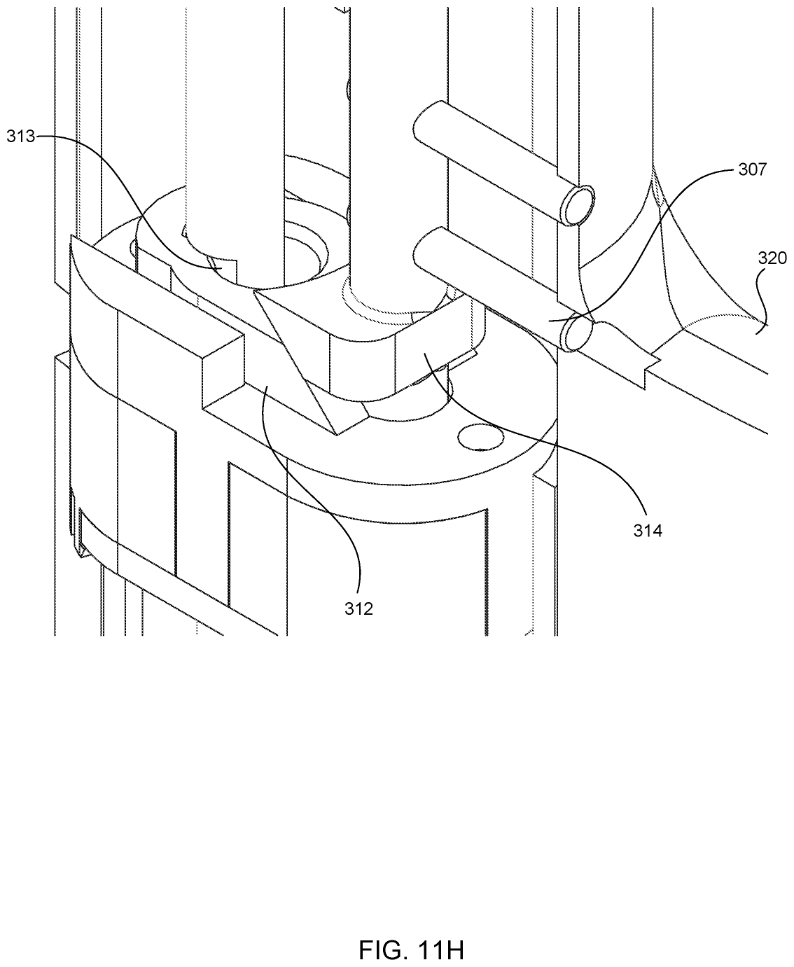

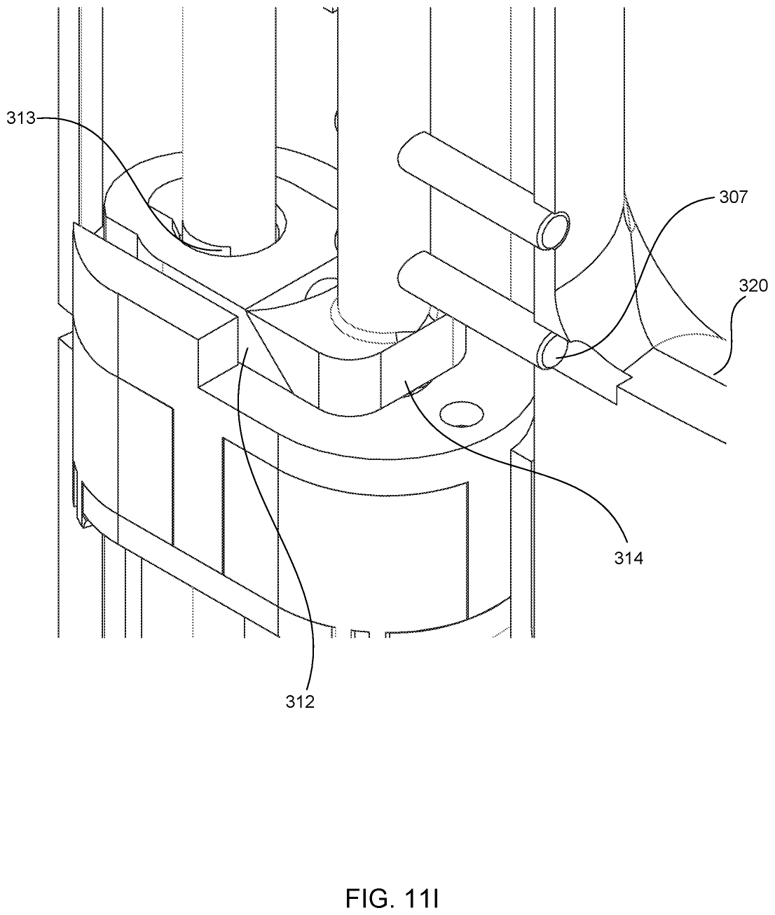

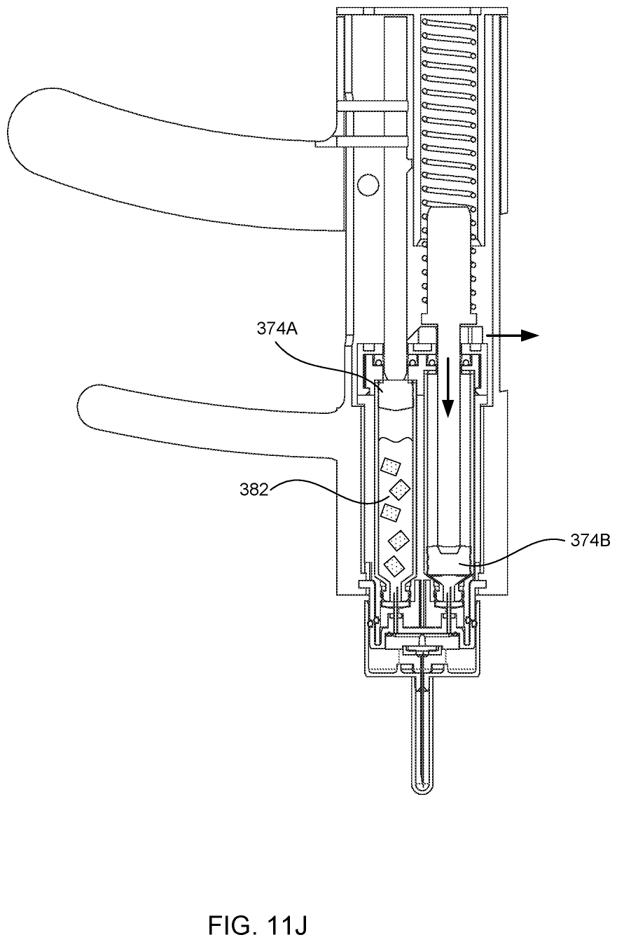

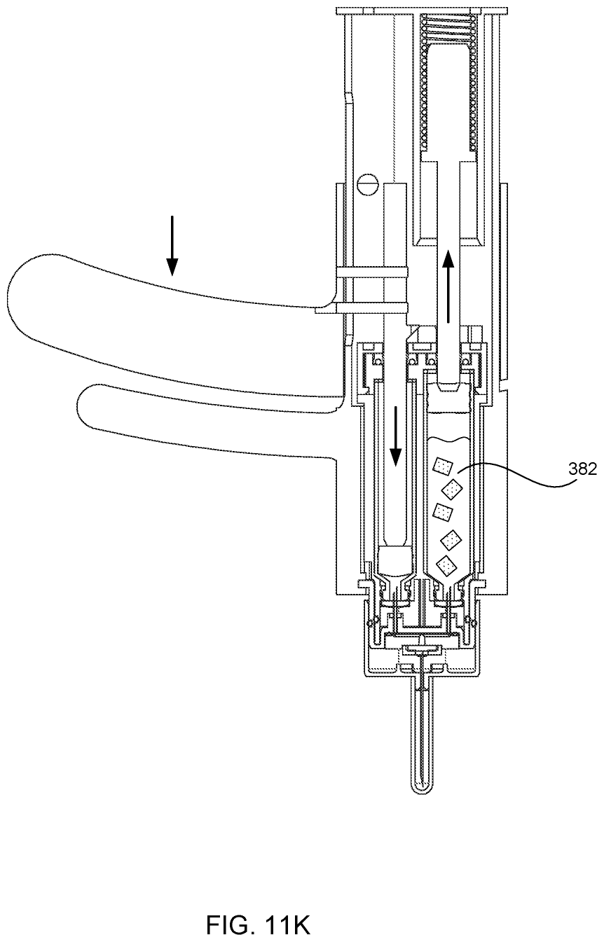

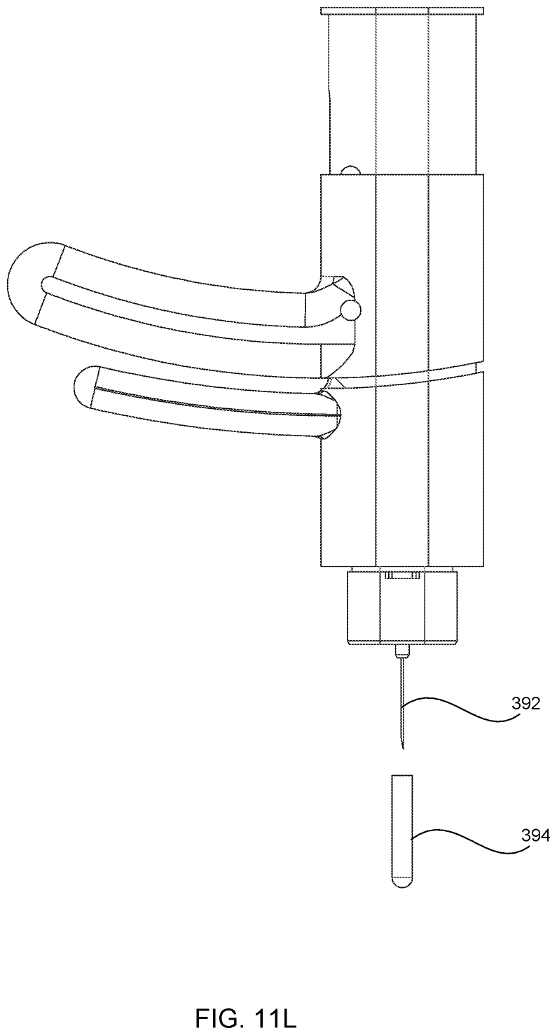

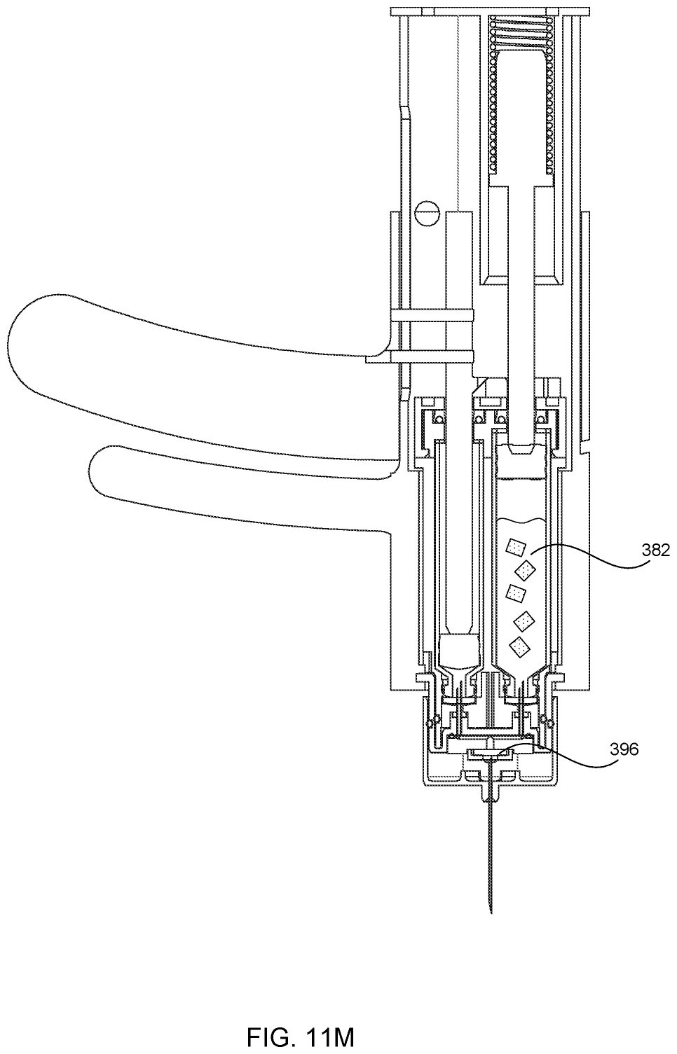

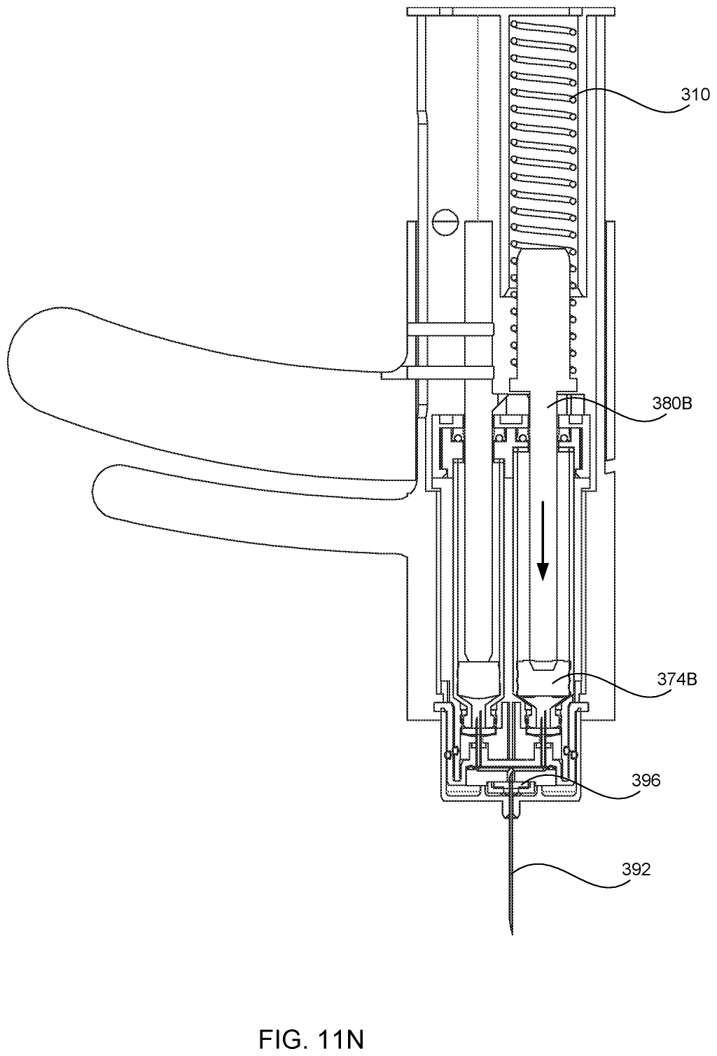

FIGS. 11A-C illustrate various views of an alternative embodiment of an inline hand compression reciprocating mixing and injector system 300. System 300 is comprised of a housing 302, a safety/activation release pin 306, a needle delivery assembly 397, a needle sheath 394, and mixing grips 320 and 321, where mixing grip 320 is a movable mixing grip and 321 is a stationary or non-moving mixing grip.

FIG. 11D illustrates a cross-sectional perspective view of the inline hand compression reciprocating mixing and injector system 300. Connecting posts 307 directly couple the movable mixing grip 320 to the plunger rod 380A. The safety pin 306 interferes with one of the connecting posts 307 to prevent it from moving until the pin 306 is removed. A compressing spring 310 acts on plunger rod 380B. Plungers 374A-B are respectively disposed below plunger rods 380A-B and disposed respectively in containers 364A-B, which are held in place by cartridge container frame 360. A fluid communication assembly 350 having mixing needles 354 and a fluid communication channel 356 is situated below 360 and is initially in a non-fluid communication state during storage. A needle assembly 397 is positioned below 350 and is also configure to be initially in a non-fluid communication state until compressed during the delivery phase. A needle sheath 394 is disposed over the delivery needle 392 until being removed for injecting.

FIG. 11E illustrates the removal of a safety/activation release pin 306 of the inline hand compression reciprocating mixing and injector system 300, which enables the mixing grip 320 to be compressed into mixing grip 321. Similar to system 200, and as shown in FIGS. 11F-G, when the user initially compresses grips 320 and 321 together, the directly coupling of grip 321 to the plunger rod 380A cause the plunger rod to push on the plunger 374A, which compresses against the liquid medicament component 381A in the first container 364A. As a result of the incompressible nature of most fluids, the force acts on the cartridge container frame 360 to drive the first and second containers 364A-B into the fluid communication assembly 350 and particularly into the mixing needles 354 to create a fluidic flow path between the first and second containers. Once the flow path is established, the continuing compression force imparted onto the mixing grips causes the medicament component 381A into the second container 364B to mix with medicament component 381B and form a mixed medicament 382.

In order to release the stored energy in the compression spring 310, the rod sliding lock 312 needs to laterally shifted or transitioned. This transition is illustrated in FIGS. 11H-I. A sliding lock engagement flange 314 is positioned along a portion of plunger rod 380A and when it has traveled sufficiently engages with a ramped portion of the rod sliding lock 312, which downward force on the ramp generates a lateral movement or shifting in 312. These shifting releases the notched portion 313 of plunger rod 380B from a ledge portion of 312 to be released and freely travel. One of the advantages of these rod sliding locks 212, 312 is that with each compression of the mixing grips ensures the plunger rods 280B, 380B are free to move. Once the rod sliding lock 312 is out of an interference position, the compression spring 310 can now act to drive the plunger rod 380B onto plunger 374B and transfer the mixed medicament from container 364B into container 364A.

FIGS. 11J-K illustrate various transfer states between containers once the spring-driven plunger rod 380B is activated and the compression spring 310 free to drive it. Similar to system 200, with each compression there is a transfer and with each release there is a transfer of mixed medicament.

When sufficient mixing has occurred, the user can prepare the device to deliver the mixed medicament 382, such as shown in FIGS. 11L-N which illustrate various phases of preparing the inline hand compression reciprocating mixing and injector system 300 to deliver and delivering a mixed medicament. The sheath 394 can be removed as shown in FIG. 11L. The user while compressing the grips, can inject the exposed delivery needle into an injection site. This injecting causes a pressure on the needle 392 and the needle assembly 390, which moves upward or distally into the fluid communication assembly 350 and pierces the delivery septum 396. Once that is accomplished the compression spring 310 drives plunger 380B to force the mixed medicament now in container 364B out of the system through the delivery into the recipient.

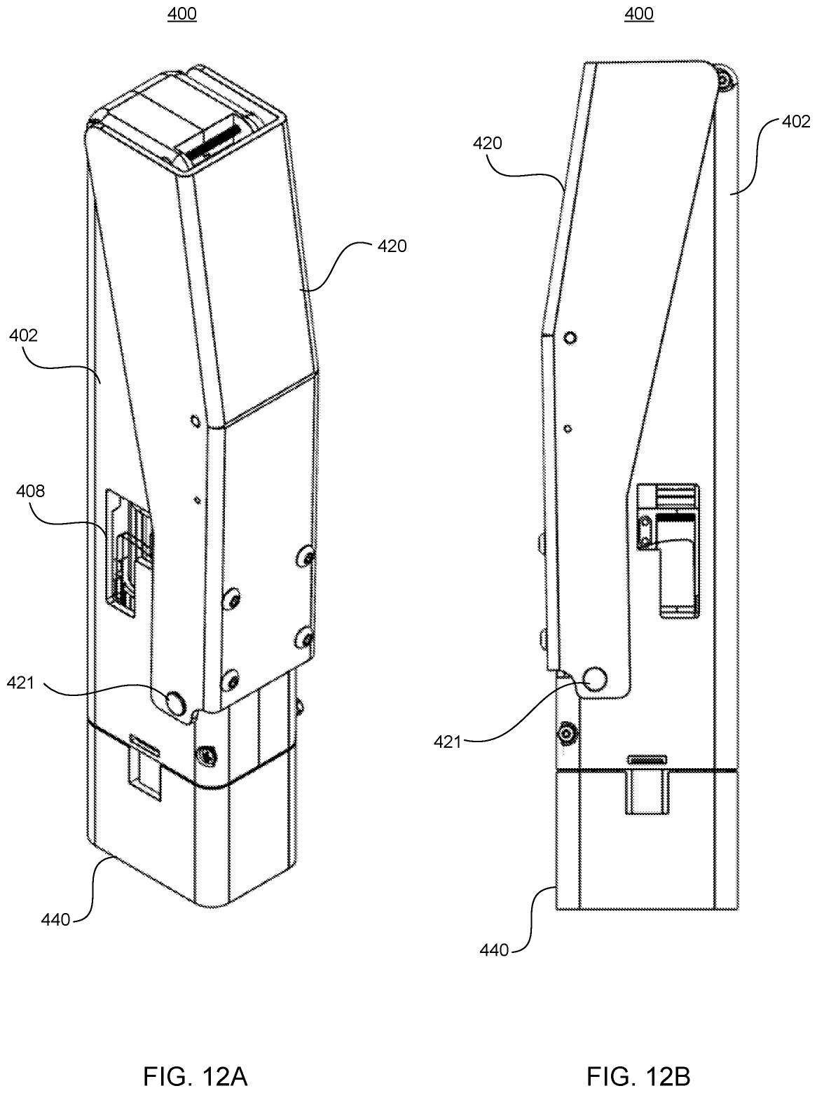

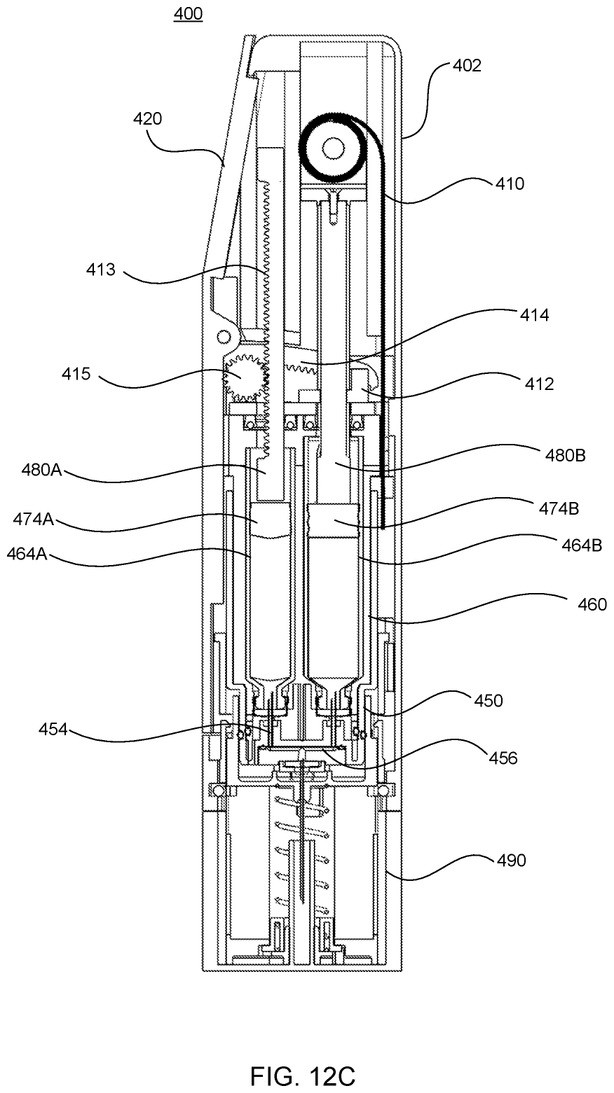

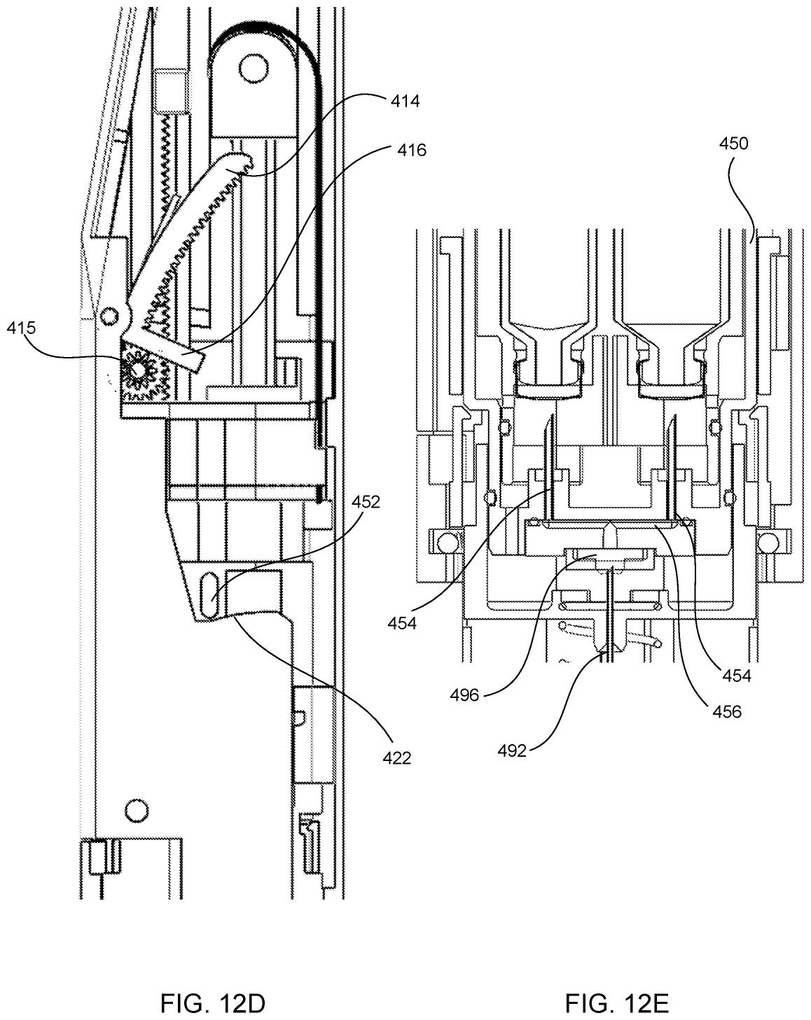

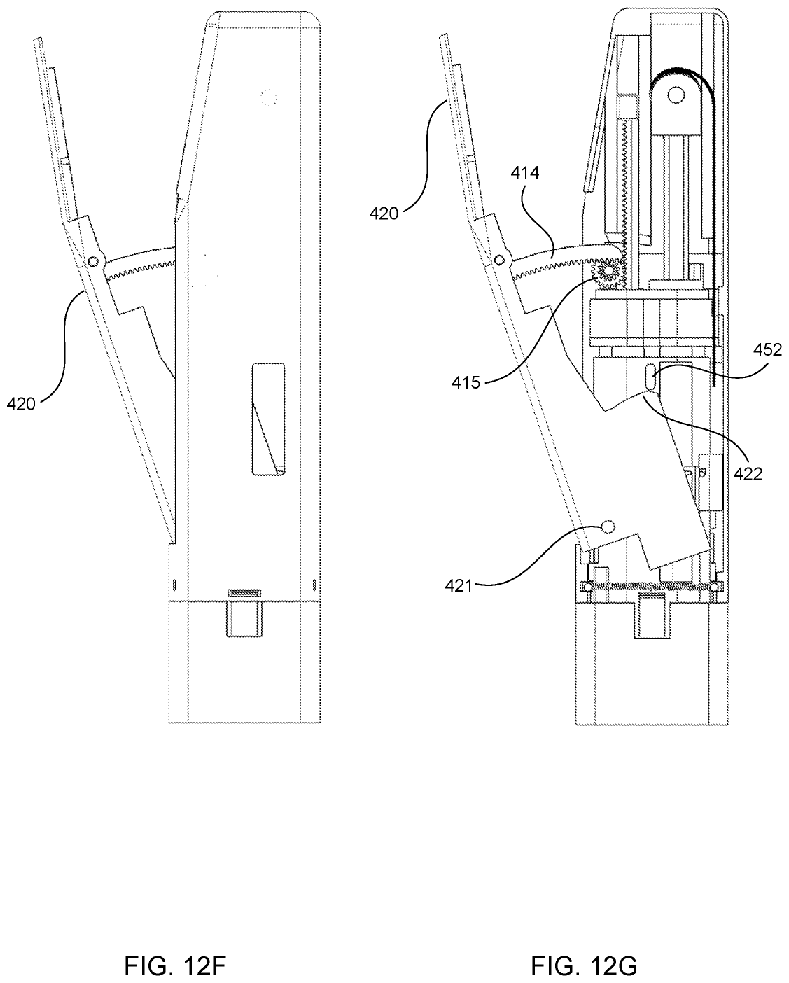

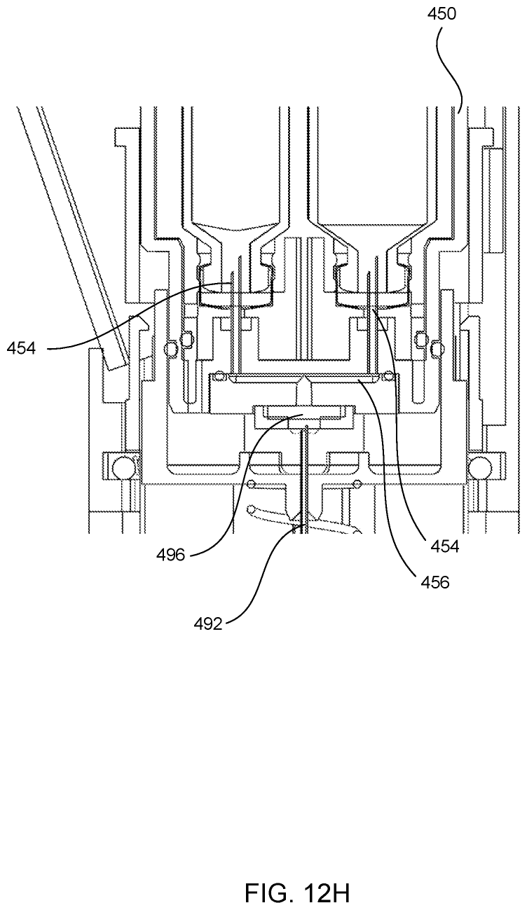

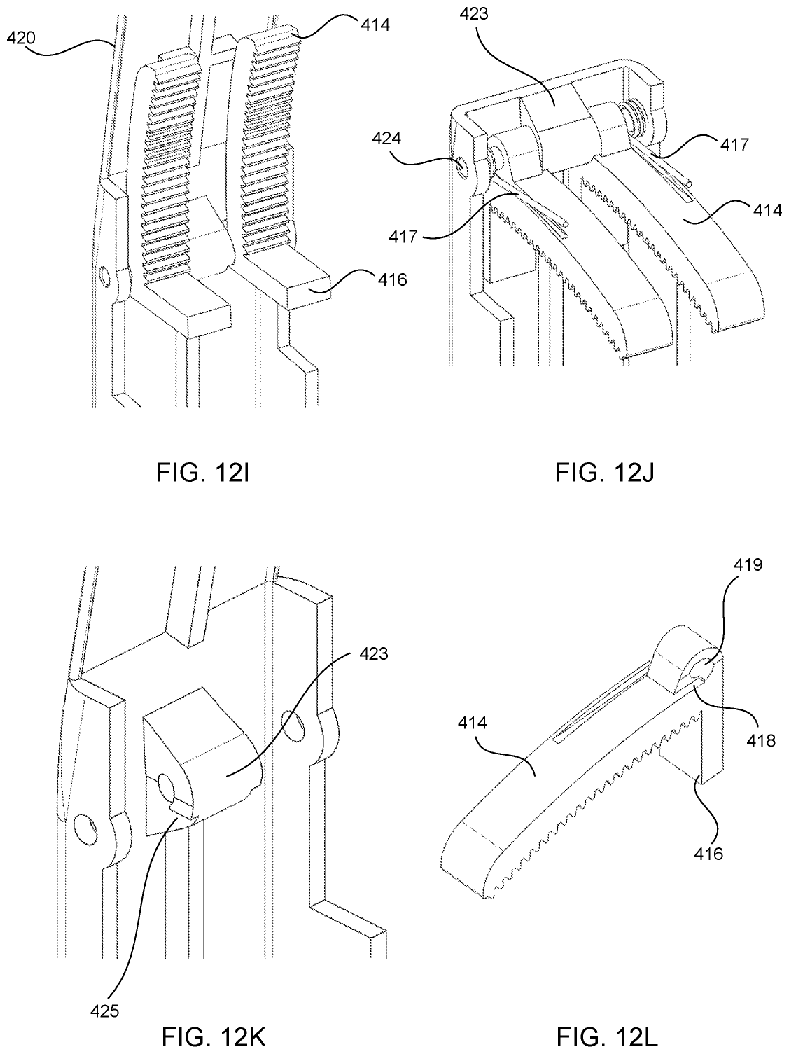

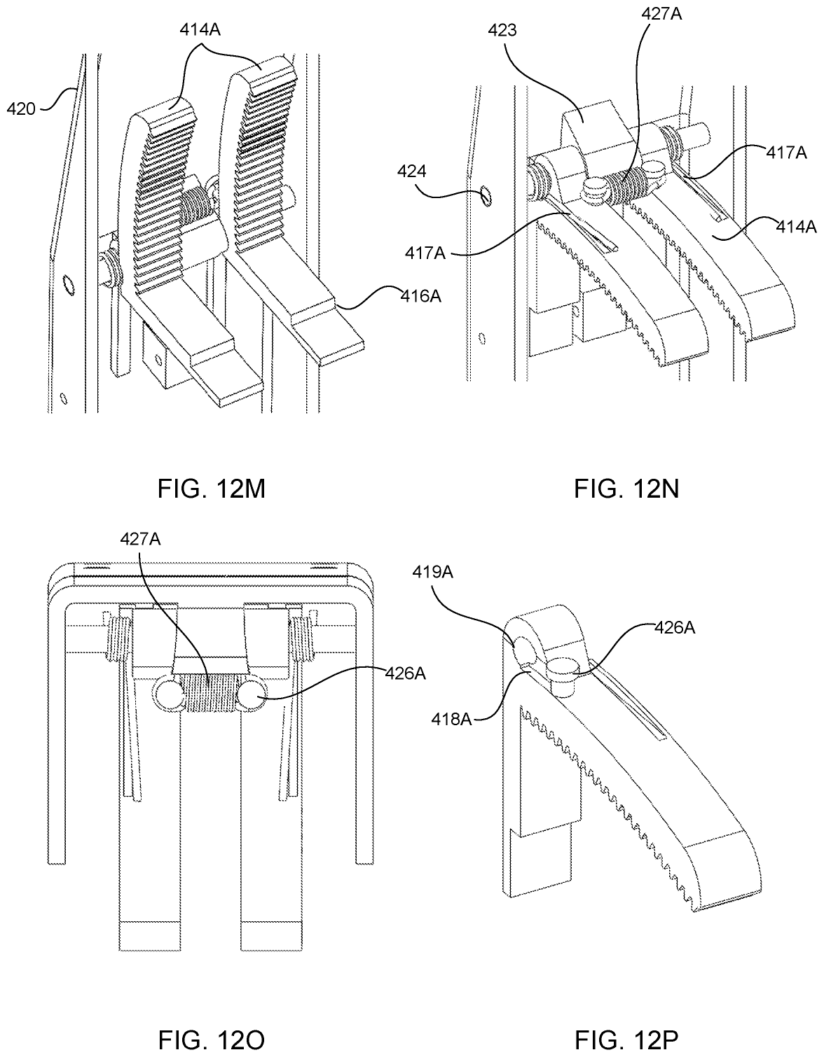

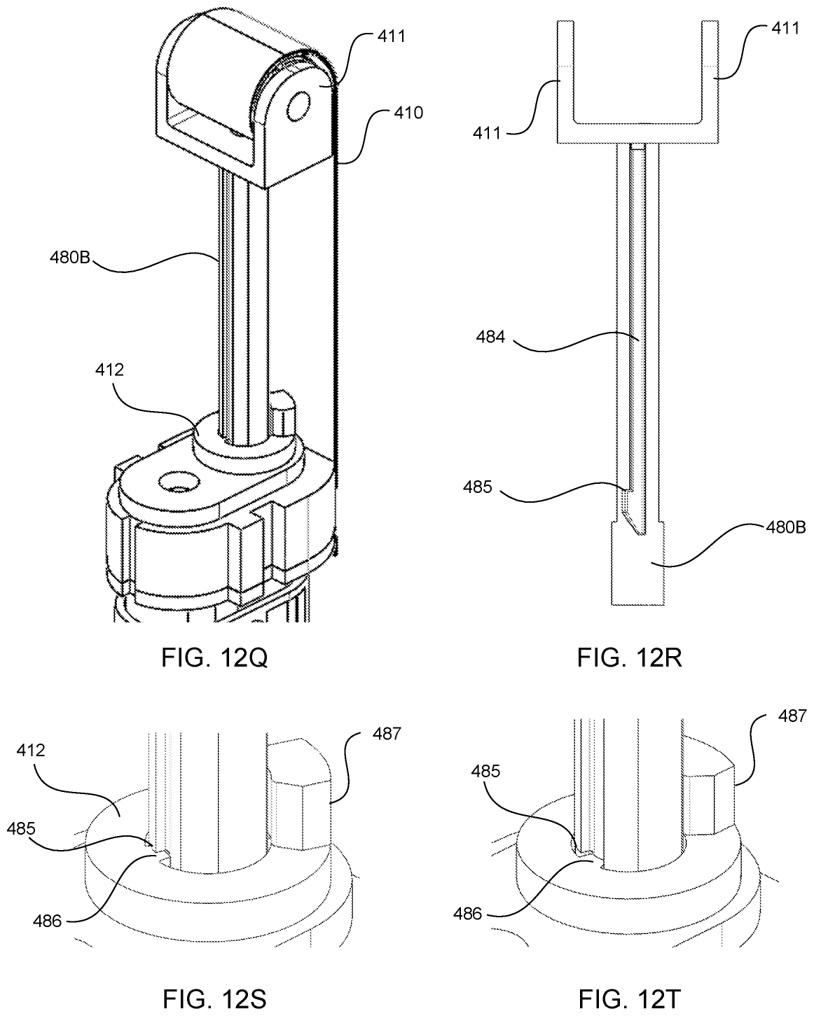

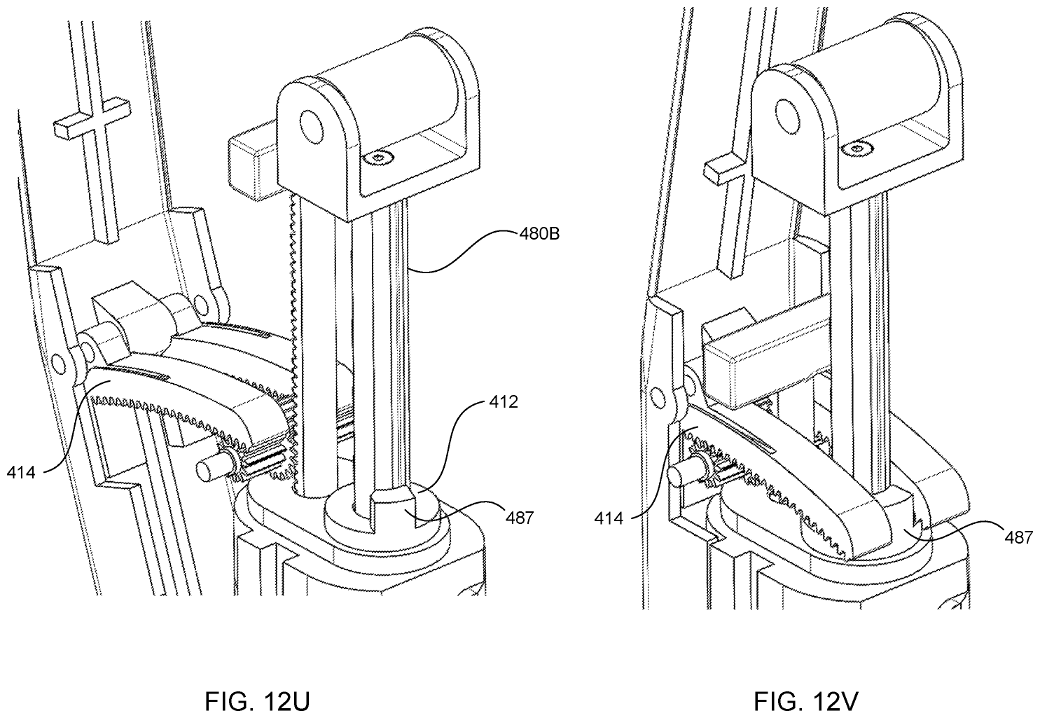

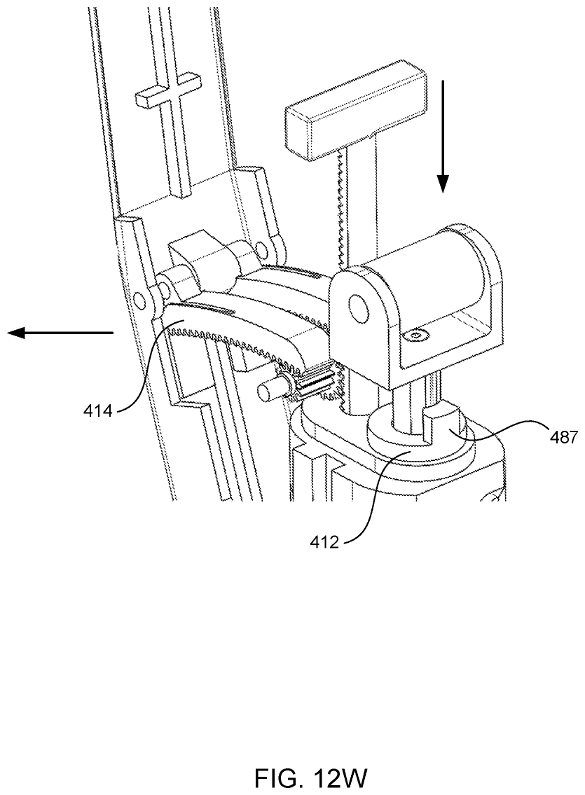

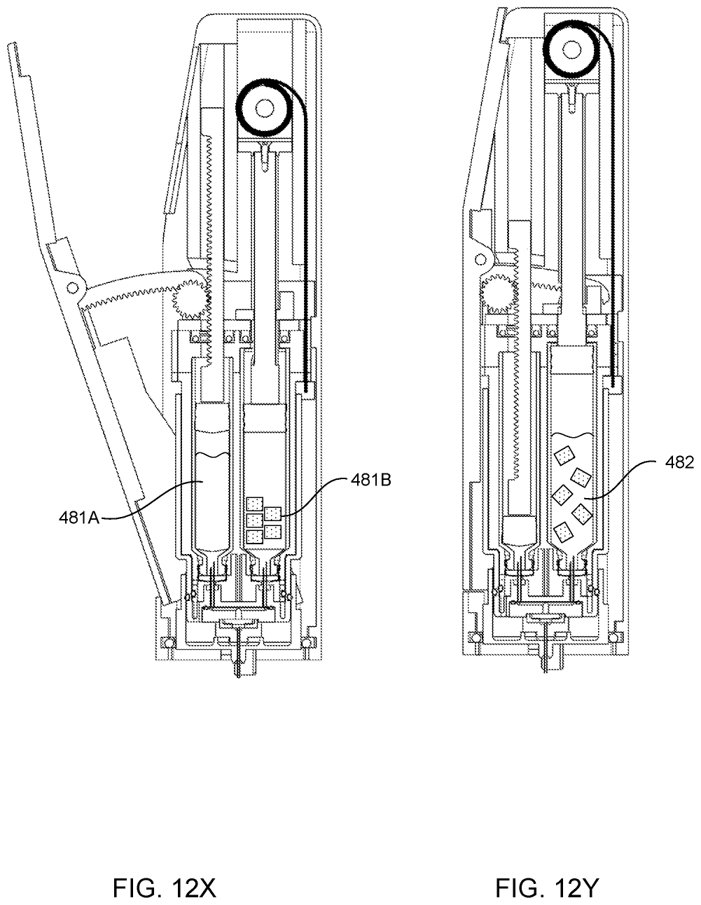

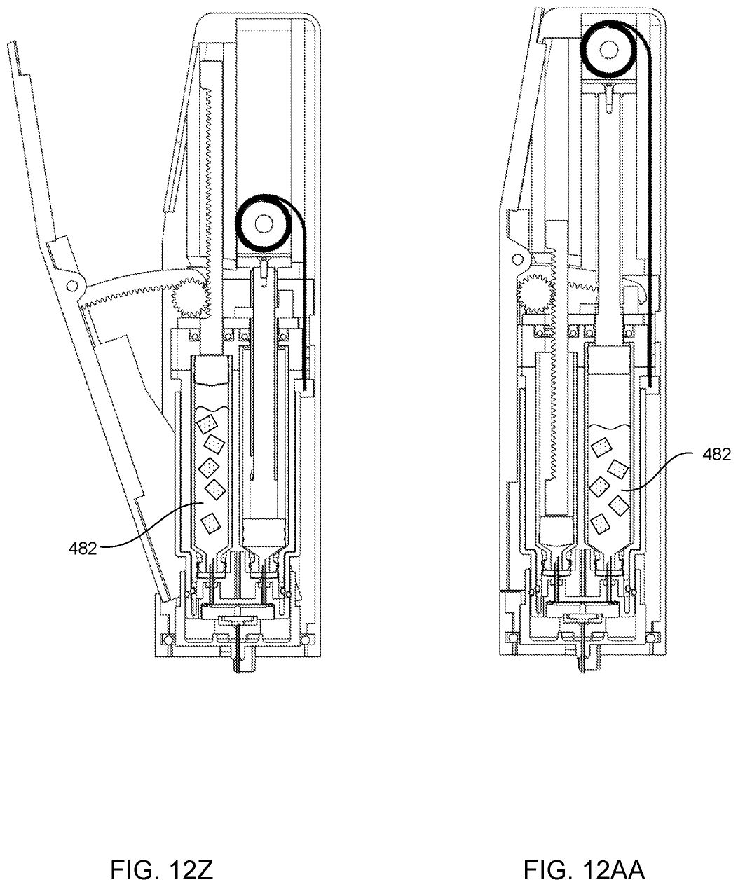

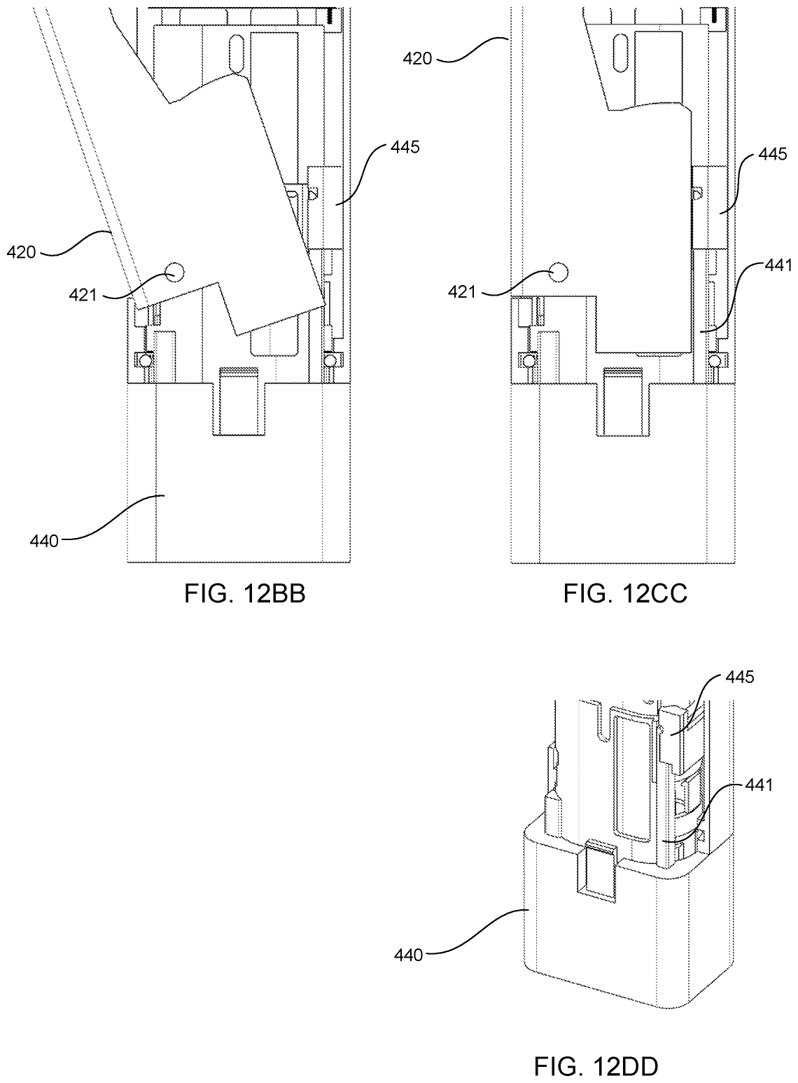

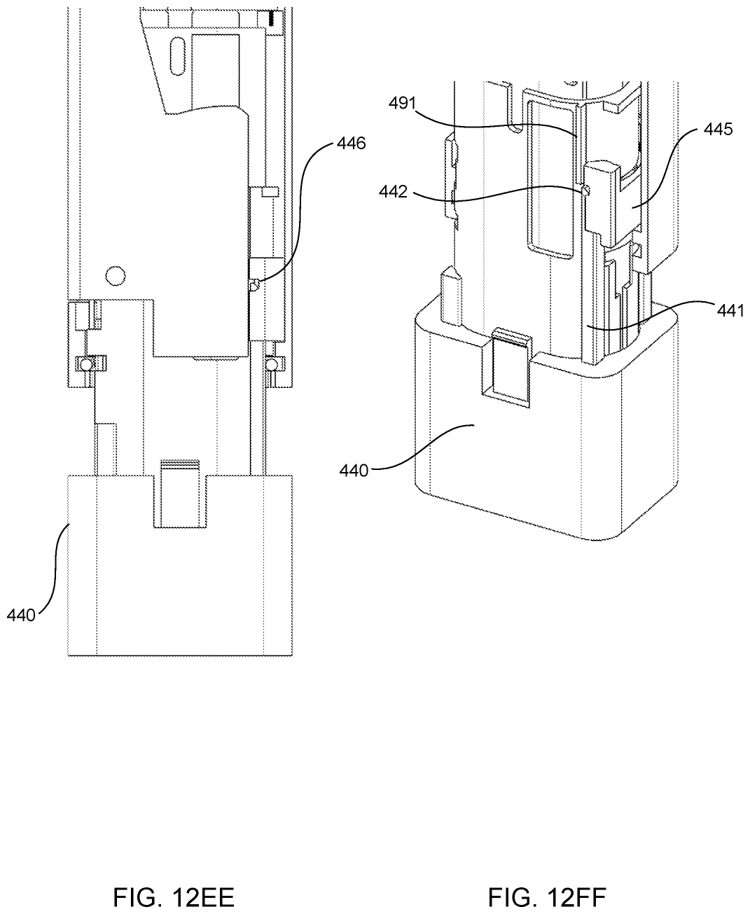

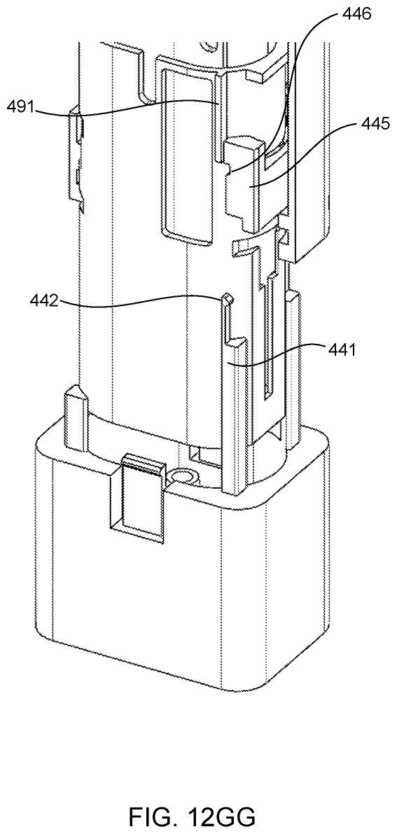

FIGS. 12A-B illustrate various views of yet another alternative embodiment of a reciprocating mixing and injector system 400 using compression lever with a rack and pinion system. System 400 includes a housing 402 having an aperture 408, a lever 420, pivoting about a pivot pin 421, and a safety cap 440.

FIG. 12C illustrates a cross-sectional view of the compression lever reciprocating mixing and injector system 400 to further illustrate the several components that enable this embodiment to store, mix and deliver a mixed medicament component 482 formed of first and second medicament components 481A-B. These include a constant force spring 410 configured to drive plunger rod 480B that drives plunger 474B into container 464B. The lever 420 has a horizontal rack 414 connected thereto that interfaces with a pinion gear 415, which is configured to drive a vertical rack 413 that is directly coupled to a plunger rod 480A that can drive a plunger 474A into container 464A. The containers 464A-B are disposed within a cartridge container frame 460. A fluid communication assembly 450 is configured to be driven up or distally into the containers 464A-B to create fluid communication between each container and is comprised of mixing needles 454 and a fluidic communication channel 456. A needle shield assembly 490 is disposed over a delivery needle 492.

FIGS. 12D-E illustrate various partial cutaway and cross-sectional views of the system 400 in a stowed state. As shown, in FIG. 12D, the horizontal rack 414 is initially stored in an upright manner having its lower leg portion 416 resting on pinion gear, but not engaged with pinion gear 415. Also shown is a fluid communication assembly protrusion 452 that interfaces with a camming edge 422 of lever 420. When the lever 420 is initially extended away from the housing 402, the horizontal rack 414 drops down and engages the pinion gear 415 and the camming edge 422 applies an upward or distal force on the protrusion 452, which causes the fluid communication assembly 450 to move upwards or distally into the containers 464A-B, where the mixing needles 454 pierce seals on the containers and create fluid communication between the two containers 464A-B. FIG. 12E illustrates a closer view of the fluid communication assembly 450 prior to engaging the containers 464A-B. Also labeled in FIG. 12E are the delivery septum 496 which separates fluid communication with the delivery needle 492 until the distal end of the delivery needle pierces the septum and comes in fluid communication with fluidic channel 456.

FIGS. 12F-H illustrate various views demonstrating creating fluid communication between the containers and activating the reciprocating mixing system, as it illustrates camming edge 422 forcing protrusion 452 upwards or distally as just noted. Horizontal rack 414 has rotated downward so that the teeth of horizontal rack 414 engage the teeth of pinion gear 415. FIG. 12H specifically illustrates a close-up view of fluid communication assembly 450 engaging with the containers.

FIGS. 12I-L illustrate various views of one embodiment of the horizontal rack components for use with the compression lever reciprocating mixing and injector system 400. In this embodiment, 414 is shown in FIG. 12I in an upright position during a stowed state. When the lever 420 is extended or pivoted away from housing 420, the lower leg portion 416 is allowed to rotate off of pinion gear 415 and the combination torsional and compression springs 417 further force horizontal rack 414 to rotate downward or proximally. The horizonal racks 416 rotate about a rack mounting pin 424 that is disposed through an alignment aperture 419 of each horizontal rack 414, sidewalls of 420 and the rack alignment and mounting protrusion 423. Once the horizontal racks have rotated to engage the pinion gear, the springs 417 push each of the horizontal racks 414 toward the protrusion 423, which includes a ledge 425 on each side that interfaces with a complementary ledge 418 of horizontal rack 416 to prevent the horizontal racks from rotating upward. This acts as a secondary mechanism to ensure the horizontal rack has a constant engagement with the pinion gear regardless of orientation.

FIGS. 12M-P illustrate various views of an alternative embodiment of the horizontal rack 414A components for use with the lever 420 of the reciprocating mixing and injector system 400. The primary differences between 414 and 414A are that 414A includes a spring post 426A that is configured to have a compression spring 427 attached thereto. With the 414A embodiment, a torsional spring 417 still helps rotate the horizontal rack 414A, but the compression spring 427A is what pulls the horizontal racks together to mount and interface with the rack alignment and mounting protrusion 423. Aside from the spring post 426A and compression spring 427A, the two variations of horizontal racks 414 and 414A operate in the same manner.

FIGS. 12Q-T illustrate various views and states of a rotary rod lock 412 for use with the reciprocating mixing and injector system 400. The rotary rod lock 412 prevents plunger rod 480B from traveling downward or proximally when the rotary rod lock 412 is in a locked position. The constant force spring 410 is mounted to the arms 411 of plunger rod 480B on one end and is grounded or fixed to the housing 402 on the opposite end. This spring 412 is constantly providing a downward force onto the plunger rod 480B. FIG. 12R isolates plunger rod 480B and shows a keyed slot 484 and keyed slot ledge 485. It is this keyed slot edge 485 that rests on the rotary lock key 486 of rotary rod lock 412, as shown in FIG. 12S until it is rotated away that the ledge 485 falls off the key 486, as show in FIG. 12T and the rotary lock key 486 is now aligned with the keyed slot 484 to freely move vertically up and down or distally and proximally in the system 400.

FIGS. 12U-W illustrate various views demonstrating how to rotate and unlock the rotary rod lock to release the plunger rod 480B. After the lever 420 is extended and the horizontal racks 414 drop to engage the pinion gear 415, the lever 420 can be compressed. This compression now transfers a force from the horizontal racks into the pinion gear 415, which drives the vertical rack 413 to also drive the plunger rod 480A it is directly coupled to. By driving the plunger 480A downward it causes the medicament component 481A currently in container 464A to transfer over into container 464B, to then mix with the medicament component 481B to form mixed medicament 482. As this is occurring the horizontal racks 414 are extending into the housing and interfacing with the rotary rod lock 412. As shown in the partially isolated components view in FIG. 12U the rotary lock protrusion 487 of rotary rod lock 412 is offset such that one of the 414 racks are able to engage it and cause 412 to rotate into a position as shown in FIG. 12V. At this position in FIG. 12V the key 486 moves into the keyed slot and out from under the ledge 485, which enables the plunger rod 480B to move up and down vertically. Now that the constant force spring can further act on 480B, when the user releases the lever 410, the constant force spring 420 drives the plunger 480B downward to engage plunger 474B, which drives the mixed medicament 482 in 464B through the fluid communication assembly 450 into container 464A, which causes plunger 474A to push upwards on plunger rod 480A, which is coupled to vertical rack 413, that now causes pinion gear 415 to rotate in a manner that puts a force on horizontal racks 414 to push lever 410 away from the housing 402. Now the reciprocating medicament transfer system is fully operational, such that with each compression of the lever 420 transfer is made from one container to the other, and with each release of the lever the constant force spring causes a transfer back from container 464B into 464A.

This transferring medicament between containers is further illustrated in FIGS. 12X-AA, which illustrate various views demonstrating the various phases or positions of the medicament components are in from a ready-to-mix phase to a ready-to-be delivered phase. FIG. 12 X illustrates the ready-to-mix phase or state of system 400. Here, as noted above, the lever 420 has been extended, which creates fluid communication between the containers 464A-B, and also creates a mechanical engagement of lever 420 via horizontal racks 414 with the pinion gear 415. When the user then compresses the lever 420 for the first time, energy is transferred into the system that drives the plunger rod 480A downward, also as noted above, which causes the plunger 474A to transfer the medicament component 481A to transfer into container 464B and become mixed with medicament component 481B to form mixed medicament 482, as shown in FIG. 12Y. At this point the rotary rod lock 412 has released plunger rod 480B. When the user releases their grip on lever 420 the constant force spring 410 now drives plunger rod 480B downward, also as noted above, which transfers the mixed medicament 482 from container 464B into container 464A, as shown in FIG. 12Z. The user can then compress the lever 420 again where mixed medicament 482 is transferred back into container 464B, as shown in FIG. 12AA. This transferring back and forth can continue until the user is satisfied the mixed medicament has been thoroughly mixed or blended, which can be in part determined by viewing the medicament through the housing aperture 408 or some predetermined number of counts. Once the user is ready to deliver the medicament they can lock the lever 420 in place. This is illustrated in FIGS. 12BB-GG demonstrating the elements and configurations that enable 420 to be locked into place prior to delivering the mixed medicament. A partial isolated view of various components is shown in FIGS. 12BB-CC, which shows when the sliding lock 445 is in an upward or distal position that the lever 420 is allowed to pivot about pivoting pin 421 and sliding 445 doesn't interfere with that pivoting. FIG. 12DD shows an isolated parts perspective view to show how an extension arm 441 of the safety cap 440 engages with the sliding lock 445.

As the safety cap 440 is pulled off or away from the housing 402, the extension arm 441 pulls down the sliding lock 445 through the extension clip 442 and sliding lock notch 446 interface. The extension clip 442 rests in the sliding lock notch 446 of 445 as it is sandwiched between 445 and a protrusion 491 extending from the needle shield assembly 490. This sandwiching prevents the extension clip 442 from being released from the sliding lock notch 446. However, as the safety cap is pull off it pulls the notch 446 past the protrusion 491, which then allows the clip 442 to disengage from the sliding lock 445. FIG. 12EE shows the sandwiching of the clip 442 and FIG. 12FF illustrates the clip moving downward beyond the protrusion 491 where it can be released. FIG. 12GG illustrates the clip 442 being released, so the safety cap 440 can be completely removed. Once this is done the system 400 is fixed in a state, such as shown in FIG. 12AA where the plunger rod 480A is completely depressed, except this time, the lever 420 cannot be extended. As a result, when the delivery needle pierces the delivery septum the force from constant force spring 410 continues to act on plunger rod 480B, which now drives plunger 474B to act on the mixed medicament 482 currently in container 464B to forced out of container 464B through the fluid communication assembly 450 and out through the delivery needle 492 into a recipient. The force causing the delivery needle 492 can occur in several ways, which are known and previously described including a direct force on the delivery needle or delivery needle assembly, a force on the needle shield assembly, which helps drive the distal end of the delivery needle through the delivery septum and other methods known. Thus, the system is configured to automatically inject the mixed medicament into a recipient using the energy provided by the force of the constant force spring, which during the delivery can no longer act on the plunger rod 480A, which is now in a locked position.

As a result of the embodiments conveyed above, it should be understood that some of the additional advantages of the systems provided herein, allow for a convenient reciprocating transfer of medicament components between cartridges or containers until the user is ready to deliver those components. The counteracting forces from the user input in the latter three embodiments, can also be redirected to become the delivery force for the mixed medicament. The mutli-directional valve and vent obstruction components of the initial embodiment disclosed in a similar manner help direct energy from the gas chamber in a particular to also deliver the mixed medicaments into the user. This ease of reciprocating and transferring medicaments and redirecting of energy of the energy sources provided to aid in the delivery of the medicament components are some of the improvements over the current state of the art and facilitate the mixing of difficult to mix medicament components as previously noted.

It should also be understood that the systems can be designed to explicitly be delivered from a particular container or designed to be delivered from the container the current mixed medicament resides or from both containers simultaneously. It should also be noted that the system can include wet and dry, as well as wet and wet medicament components.

It should be noted the size of the containers can be the same or they can vary in size. For example, a 3 mL and 5 mL or two 3 mL containers could be used. However, this invention and these embodiments should not be limited to these particular sizes alone and these provided as examples.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

1. A mixing and drug delivery system comprising:
a housing configured to hold a first container and a second container, where in the first container contains a first medicament component and the second container contains a second medicament component;
a first seal associated with the first container;
a second seal associated with the second container;
a mixing activation mechanism;
a fluid communication assembly having a fluidic channel configured to receive a first output from the mixing activation mechanism, whereupon receiving the first output from the mixing activation mechanism causes the fluid communication assembly to open, remove or otherwise pierce the first seal and the second seal and create a fluidic pathway between the first container and the second container;
a mixing system configured to alternately transfer the first and second medicaments between the first and second containers during a mixing phase;
a pressurized gas chamber at least partially disposed in the housing and configured to receive a second output from the mixing activation mechanism, whereupon receiving the second output causes the pressurized gas chamber to pressurize the mixing system;
a mixing trigger configured to release a portion of pressurized gas that facilitates the transfer of the first and second medicaments components between the first and second containers by the mixing system, wherein the transfer between first and second containers causes the first and second medicament components to become a mixed medicament; and
a needle delivery assembly configured to be in fluid communication with the first and second containers during a delivery phase.

2. The mixing and drug delivery system of claim 1, wherein the housing is formed in a T-shape, and wherein the lower portion or shaft portion of the T-shape forms a handle.

3. The mixing and drug delivery system of claim 1, wherein the mixing activation mechanism partially encloses the pressurized gas chamber.

4. The mixing and drug delivery system of claim 1, wherein the mixing system further comprises a first gas-driven plunger associated with the first container and a second gas-driven plunger associated with the second container.

5. The mixing and drug delivery system of claim 4, wherein the mixing system further comprises a multi-directional valve configured to alternate the flow of gas directed to the first and second gas-driven plungers based on user input to the mixing trigger.

6. The mixing and drug delivery system of claim 5, whereupon receiving the second output also causes the mixing system to initially drive the first gas-driven plunger to transfer the first medicament component from the first container into the second container with the second medicament component.

7. The mixing and drug delivery system of claim 5, whereupon a user depressing the mixing trigger causes a release of a portion of gas to drive either the first or second gas-driven plunger.

8. The mixing and drug delivery system of claim 7, whereupon the user releasing the mixing trigger causes the release of a portion of gas to drive either the first or second gas-driven plunger.

9. The mixing and drug delivery system of claim 7, whereupon each subsequent depressing of the mixing trigger by the user causes the release of a portion of gas to be alternately directed to drive either the first or second gas-driven plunger.

10. The mixing and drug delivery system of claim 8, whereupon each subsequent releasing of the mixing button by the user causes the release of a portion of gas to be alternately directed to drive either the first or second gas-driven plunger.

11. The mixing and drug delivery system of claim 1, wherein the fluid communication assembly further includes a fluidic transfer channel that fluidly connects the first container and the second container upon receiving the first output to the fluid communication assembly.

12. The mixing and drug delivery system of claim 11, further including a delivery seal configured to prevent fluid communication between the fluidic transfer channel and the needle assembly during the mixing phase.

13. The mixing and drug delivery system of claim 11, wherein the fluidic transfer channel and the needle assembly are configured to be in fluid communication, and wherein the needle assembly further includes a sterility barrier covering an injection end of an injection needle of the needle assembly.

14. The mixing and drug delivery system of claim 12, wherein the needle assembly further includes a needle shield configured to be a bump trigger, and a needle shield lockout mechanism configured to maintain the needle shield in an extended state after a delivery phase.

15. The mixing and drug delivery system of claim 14, further including a delivery actuation system having at least one stored energy and configured to drive the needle of the needle assembly into a user upon being activated by the bump trigger.

16. The mixing and drug delivery system of claim 5, wherein the multi-directional valve includes a vent associated with each of the first and second gas-driven plungers and configured to release pressure from either the first or second gas-driven plunger when a new portion of gas released is directed at the alternate of the first and second gas-driven plungers.

17. The mixing and drug delivery system of claim 16, further including at least one vent obstruction component.

18. The mixing and drug delivery system of claim 17, further including a vent lockout mechanism configured to move the at least one vent obstruction component in a position to block the flow of gas from exiting one of the vents of the multi-directional valve.

19. The mixing and drug delivery system of claim 18, wherein the vent lockout mechanism includes a slide actuator having at least one ramped protrusion configured to interface with the at least one vent obstruction component.

20. The mixing and drug delivery system of claim 18, wherein the vent lockout mechanism includes a camming component configured to interface with the at least one vent obstruction component.

21-99. (canceled)
